(19) United States
(12) Patent Application Publication
(10) Pub. No.: US 2003/0216339 A1
(43) Pub. Date: Nov. 20, 2003

(54) Gene sequences associated with neural plasticity and methods related thereto

(75) Inventors: Max Cynader, West Vancouver (CA); Shiv Prasad, Vancouver (CA)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Max Cynader, Vancouver (CA)

(21) Appl. No.: 10/355,716

(22) Filed: Jan. 31, 2003

Related U.S. Application Data
(63) Continuation of application No. 09/301,507, filed on Apr. 28, 1999.
Application No. 09/301,507 is a continuation of application No. 08/224,621, filed on Apr. 5, 1994, now abandoned.

(51) Int. Cl. C12Q 1/68; C07H 21/04; A61K 48/00; C12N 5/08

(52) U.S. Cl. 514/44; 536/23.2; 435/6; 435/368; 424/93.21

(57) ABSTRACT

The disclosed invention generally relates to newly identified polynucleotide sequences capable of conferring neural plasticity to neurons. Additionally, this application encompasses the complete gene sequences, the polypeptides associated therewith, and methods of using. These methods include the use of the sequences for gene therapy, including the treatment or cure of genetic diseases, for the treatment of degenerative disorders, for the treatment of neuron damage, and for the treatment of learning disorders. Additionally, these sequences are useful to enhance memory and learning capacity.

### TECHNICAL FIELD

The present invention relates to newly-identified polynucleotide sequences capable of conferring neural plasticity, and to the complete gene sequences and polypeptides associated therewith and to uses thereof.

### BACKGROUND OF THE INVENTION

Identification and sequencing of genes is a major goal of modern scientific research. By identifying genes and determining their sequences, scientists have, for example, been able to make large quantities of valuable human “gene products.” These include human insulin, interferon, Factor VIII, tumor necrosis factor, human growth hormone, tissue plasminogen activator, and numerous other compounds. Additionally, knowledge of gene sequences of the central and peripheral nervous systems can provide the key to treatment or cure of genetic diseases, degenerative disorders, neural damage or regrowth and learning disorders.

Genes are the basic units of inheritance. Each gene is a string of connected bases called nucleotides. Most genes are formed of deoxyribonucleic acid, DNA. (Some viruses contain genes of ribonucleic acid, RNA.) The genetic information resides in the particular sequence in which the bases are arranged. A sequence of nucleotides is often called a polynucleotide or an oligonucleotide. A triplet of nucleotides, called a “codon,” in DNA codes for each amino acid or signals the beginning or end of the message, called an “anticodon.” The term codon is also used for the corresponding (and complementary) sequences of three nucleotides in the mRNA into which the original DNA sequence is transcribed.

Like genes, polypeptides are built from long strings of individual units. These units are termed “amino acids.” The polynucleotide of a gene tells the cell the sequence in which to arrange the amino acids to make the polypeptide encoded by that gene. In general, chains of up to about 200 amino acids are called “polypeptide subunits” or “polypeptides.”

Generally, enzymes in the cell transcribe the DNA of the gene into a transient RNA copy, called messenger RNA or mRNA. The mRNA, in turn, can be translated into a polypeptide by the cell. This entire process is called “gene expression,” and the polypeptide is the “gene product.” Scientists have discovered how to reverse the transcription process and copy mRNA back into DNA using an enzyme called “reverse transcriptase.” The resulting sequence is called “complementary DNA” or “cDNA.” When substantially all of the mRNA from a cell or a tissue is converted to cDNA and cloned into multiple copies of a recombinant vector to allow replication and manipulation in the laboratory, the resulting series of sequences are referred to collectively as a “cDNA library.”

The various types of genes include those which code for polypeptides, those which are transcribed into RNA but are not translated into polypeptides, and those whose functional significance does not demand that they be transcribed at all. Most genes are found on large molecules of DNA located in chromosomes. Double-stranded cDNA carries all the information of a gene. Each base of the first strand is joined to a complementary base, or “hybridized,” in the second strand. The linear DNA molecules in chromosomes have thousands of genes distributed along their length. Chromosomes include both coding regions, which code for polypeptides, and noncoding regions; the coding regions represent only about three percent of the total chromosome sequence.

An individual gene has regulatory regions including a promoter that directs the expression of the gene, a coding region that may code for a polypeptide, and a termination signal. The regulatory DNA sequence is usually a noncoding region that determines if, where, when, and at what level a particular gene is expressed.

The coding regions of many genes are discontinuous, with coding sequences, or “exons,” alternating with noncoding regions, or “introns.” The final mRNA copy of the gene does not include these introns (which can be much longer than the coding region itself), although it does contain certain untranslated regions that usually do not code for the polynucleotide gene product. Untranslated sequences at the beginning and end of the mRNA are known as 5′- and 3′-untranslated regions, respectively. This nomenclature reflects the orientation of the nucleotide constituents of the mRNA

A cDNA is a DNA copy of a messenger RNA, which contains all of the exons of a gene. The cDNA can be thought of as having three parts: an untranslated 5′ leader, an uninterrupted polypeptide-coding sequence, and a 3′ untranslated region. The untranslated leader and trailing sequences are important for initiation of translation, mRNA stability, and other functions. The untranslated leader and trailing sequences are called 5′- and 3′-untranslated sequences, respectively. The 3′ untranslated sequence is usually longer than the 5′-untranslated leader, and can be longer than the polypeptide-coding sequence. The untranslated regions typically have many, randomly-distributed stop codons, and do not display the nonrandom base arrangements found in coding sequences. The 5′-untranslated sequence is relatively short, generally between 20 and 200 bases. The 3′-untranslated sequence is often many times longer, up to several thousand bases.

The translated or coding sequence begins with a translational start codon (AUG or GUG) and ends with a translational stop codon (UAA, UGA, or UAG). Generally, translation begins at the first “start” codon on the mRNA and proceeds to the first “stop” codon. Coding sequences can be distinguished by their nonrandom distribution of bases; numerous computer algorithms have been developed to distinguish coding from noncoding regions in this way.

Human DNA differs from person to person. No two persons (except perhaps identical twins) have identical DNA. While the differences, called allelic variations or polymorphisms, are slight on a molecular level, they account for most of the physical and other observable differences between individuals. It has been estimated that approximately 14 million sequence polymorphism differences exist between individuals.

The ability of one strand of DNA to attach or hybridize to a complementary strand has already been exploited for several purposes. For example, small pieces of DNA (15 to 25 base pairs long) can be made which will hybridize to longer strands of DNA which have a complementary sequence. These short “primers” can be selected such that they hybridize to a specific, unique location on the longer strand. Once the primers have hybridized to their target on the DNA, the polymerase chain reaction (PCR) can be employed to generate millions of copies of (or amplify) the particular segment of DNA between the locations to which two primers are bound. Briefly, this technique allows amplification of a DNA region situated between two convergent primers, using oligonucleotide primers that hybridize to opposite strands. Primer extension proceeds inward across the region between the two primers, and the product of DNA synthesis of one primer serves as a template for the other primer. Repeated cycles of DNA denaturation, annealing of primers, and extension result in an exponential increase in the number of copies of the region bounded by the primers.

Similarly, a labeled segment of single-stranded DNA can be hybridized to a longer DNA sequence, such as a chromosome, to mark a specific location on the longer sequence. Segments of DNA 50 bases long or longer that hybridize to a unique DNA location in the human genome are extremely unlikely to hybridize elsewhere in the human genome. Because coding regions comprise such a small portion of the human genome, identification and mapping of transcribed regions and coding regions of chromosomes is of significant interest.

Previous studies have shown that relatively few brain mRNAs with regionally heterogeneous distributions are of sufficient abundance to permit detection of their corresponding cDNA clones by differential colony screening. Differential screening of cDNA libraries with labeled first strand cDNAs synthesized from unfractionated RNA can only detect clones representing highly abundant mRNA species (0.1% abundance or more). Current evidence indicates that many mRNA species that are known to be of biological importance (such as transcription and growth factors for instance) are present only in low abundance. Thus, detection of low abundance clones is of obvious importance particularly for use in gene therapy in delicate neural tissues.

The search for treatments for neural degenerative disorders and injury to neural tissues has been relatively unsuccessful. The term “degenerative”, as applied to diseases of the nervous system, is used to designate a group of disorders in which there is a gradual, generally symmetric, relentlessly progressive wasting away of neurons, for reasons still unknown. Many of the conditions so designated depend on genetic factors and thus appear in more than one member of the same family. This general group of diseases is therefore frequently referred to as “heredodegenerative.” A number of other conditions, not apparently differing in any fundamental way from the hereditary disorders, occur only sporadically, i.e., as isolated instances in a given family.

It is a characteristic of the degenerative diseases that they begin insidiously and run a gradual progressive course which may extend over many years. The earliest changes may be so slight that it is frequently impossible to assign any precise time of onset. However, as other gradually developing conditions, the patient may give a history implying an abrupt appearance of the disability. This is particularly likely to occur if there has been an injury, or if some other dramatic event has taken place in the patient's life, to which illness might conceivably be related. In such a case, skillfully taking of the history may bring out that the patient or family had suddenly become aware of the condition which had, in fact, already been present for some time but had passed unnoticed. Whether trauma or other stress may bring on or aggravate one of the degenerative diseases is still a question that cannot be answered with certainty. From all that is known, it would seem highly improbable that this could happen. In any event, it must be kept in mind that the disease processes under discussion, by their very nature, developed spontaneously without relation to external factors.

Family history of degenerative nervous diseases is a significant feature of this class of diseases. Another significant feature is that, in general, their ceaselessly progressive course is uninfluenced by all medical or surgical measures. Dealing with a patient with this type of illness is often, therefore, an anguishing experience for all concerned. Its symptoms can often be alleviated by wise and skillful management.

A striking feature of a number of disorders of this class is the almost selective involvement of the anatomically or physiologically related systems of neurons. This is clearly exemplified in amyotrophic lateral sclerosis, in which the process is almost entirely limited to cortical and spinal motor neurons, and in certain types of progressive ataxia, in which the Purkinje cells of the cerebellum are alone affected. Many other examples could be cited in which certain neuronal systems disintegrate, leaving others perfectly intact.

An important group of degenerative diseases has therefore been called “system diseases,” and many of these are strongly hereditary. It must be realized, however, that selective involvement of neuronal systems is not exclusively a property of the degenerative group, since several disease processes of known cause have similar circumscribed effects on the nervous system. Diptheria toxin, for instance, selectively attacks the myelin of the peripheral nerves. Another example is a special vulnerability of the PurkLnje cells of the cerebellum to hyperthermia. On the other hand, several of the conditions included among degenerative diseases are characterized by the pathologic changes that are diffuse and unselective. These exceptions nevertheless do not detract from the importance of affection of particular neuronal systems as a distinguishing feature of many of the diseases under discussion.

Learning disorders, ranging from dyslexia to mental retardation, can be equally devastating. However, in these circumstances, instead of the slow degeneration of the brain function, the afflicted party struggles with an inability to comprehend and/or retain information. The broad spectrum of learning disorders may arise as a result of heredity or injury. For example, it is known that there are specific areas of cortex in the left hemisphere of the brain that are specifically active during reading, and it is known that damage to these areas results in specific loss of reading capabilities. A review of specific cortical areas involved in reading and language is found in Maveux, E. and Kandel, E. R., Chapter 54 (pp. 839-851) in Kandel, E. R., Schwartz, J. H., and Jessell, T. M., Principles of Neural Science, 3d ed., Elsevier Press, N.Y., 1991, incorporated herein by reference.

Additional background information and definitions for scientific terms can be found in the literature. See, for example, Rieger et al., Glossary of Genetics, Classical and Molecular, 5th ed., Springer-Verlag, New York, 1991. The contents of this and other publications cited in the specification are incorporated by reference herein.

The present invention discloses novel compositions and methods for conferring neural plasticity to regions of the brain in need thereof, and further provides other related advantages.

### SUMMARY OF THE INVENTION

Briefly stated, the paresent invention provides compostions and methods for conferring neural plasticity to cells of the peripheral and central nervous systems.

One aspect of the invention includes cDNA libraries isolated from a visual cortex of a kitten about 24-35 days old.

Another aspect of the invention includes cDNA libraries comprising polynucleotides differentially expressed between polynucleotides isolated from a visual cortex of a kitten about 24-35 days and polynucleotides isolated from a visual cortex of an adult feline.

Another aspect of the invention includes cDNA libraries comprising differentially expressed between polynucleotides isolated from a visual cortex of a dark-reared adult feline and polynucleotides of a visual cortex of an adult feline.

Another aspect of the invention includes cDNA libraries comprising polynucleotides isolated from the visual cortex of a dark-reared adult feline.

Yet another aspect of the invention includes compositions comprising an isolated polynucleotide having a sequence designated as one of: SEQ. ID. NOS.: 1-132 or allelic variation thereof or complementary sequence thereto, or portion thereof at least 15 nucleotides in length.

Yet another aspect of the invention includes isolated nucleic acid molecules comprising human genes capable of hybridizing to a sequence designated as any one of SEQ. ID. NOS.: 1-93, 120-132, or to a sequence complementary thereto, under hybridization conditions sufficiently stringent to require at least about 80% base pairing.

Another aspect of the invention includes antisense polynucleotides and triple helix probes capable of blocking expression of a gene product.

Another aspect of the invention includes constructs capable of directing the expression of any one of the disclosed nucleic acid molecules of the invention.

Yet another aspect of the invention includes methods of treating warm-blooded animals for neurological disorders, by administering to a warm-blooded animal a therapeutically effective amount of a composition comprising a polynucleotide of the present invention, in combination with a pharmaceutically acceptable carrier or diluent such that said neurological disorder is treated.

Another aspect of the invention includes methods of treating learning disorders by administering a warm-blooded animal a therapeutically effective amount of a composition comprising a polynucleotide of the present invention in combination with a pharmaceutically acceptable diluent or carrier, such that the learning disorder is treated.

Another aspect of the invention includes methods of enhancing learning and memory of warm-blooded animals, comprising administering an effective amount of a polynucleotide of the present invention in combination with a pharmaceutically acceptable carrier or diluent, such that learning and memory are enhanced.

Yet another aspect of the invention includes a pharmaceutical composition, comprising any one of the polynucleotides of the present invention in a pharmaceutically acceptable diluent or carrier.

These and other aspects of the present invention will become evident upon reference to the following detailed description and attached drawings. In addition, various references are set forth which describe in more detail certain procedures and/or compositions, and are hereby incorporated by reference in their entirety.

### BRIEF DESCRIPTION OF THE DRAWINGS

FIG. 1 depicts the scheme of the strategy employed in the preparation of a 30-day-old kitten visual cortex specific subtracted probe outlined in the examples.

FIG. 2 depicts an autoradiogram showing an example of a subtractive-hybridization screening of a 30 day old kitten visual cortex cDNA library. The 12,000 cDNA clones constructed were spread on 12 Luria broth plates containing 50 μg of ampicillin oer ml at a density of 1000 clones per plate. The colonies were transferred to nylon membranes and lysed in situ. The filters were hybridized with the 30-day-old kitten visual cortex subtracted probe as described in materials and methods. The clones irrespective of their signal intensity were individually isolated.

FIG. 3 depicts northern blot analysis with RNAs (10 μg) from the 30-day-old kitten visual cortex and adult cat visual cortex probed with riboprobes derived from eight of the 200 purified cDNA clones. Hybridization was carried out with the probes indicated below; (a) pKVC6, (b) pKVC79, (c) pKVC80, (d) pKVC82, (e) pKVC90, (f) pKVC100, (g) pKVC108 and (h) pKVC110.

FIG. 4 depicts a diagram of possible cellular location of the identifiable proteins which are enriched in the 30 day kitten visual cortex.

FIG. 5 depicts sequence listings for SEQ. ID. NOS.: 1-132.

### DETAILED DESCRIPTION OF THE INVENTION

This invention is generally directed to several cDNA libraries, specific polynucleotides, allelic variations thereof or complementary sequences thereto, or portions thereof at least 15 nucleotides in length, and their associated gene sequences and polypeptides, all of which are capable of conferring the characteristic of neural plasticity. These nucleic acid sequences and polypeptides have a multitude of uses, including as markers or probes, i.e., in chromosome mapping, and for use in gene therapy.

One aspect of the present invention provides cDNA libraries constructed from feline visual cortices using any one of a variety of techniques, including, for example, a cDNA library kit available from Stratagene Corporation. Feline visual cortices are easily identifiable by observation and may be isolated and processed as set forth in the Examples below.

Those polynucleotides particularly suitable for conferring neural plasticity are then isolated from the libraries by selecting those polynucleotides which appear either uniquely, or in much greater relative abundance, in the critical stage kitten when compared to the adult feline. In the context of the present invention, these sequences are referred to as “differentially expressed sequences.” Selection of differentially expressed sequences may be accomplished using any one of several techniques, including, for example, differential screening, differential display polymerase chain reaction (“PCR”), and subtractive hybridization. See Innis et al., PCR Protocols: A Guide to Methods and Applications, Academic Press, Inc., 1990. Preferably, the sequences may be selected using any suitable technique capable of detecting polynucleotides present as low as 0.01% of the total mRNA population, including subtractive hybridization as described in detail in Sieve and St. John, Nucl. Acids Res. 16:10937, 1988, incorporated herein by reference.

In the context of the present invention, “neural plasticity” refers to the ability of a cell to make a long term alteration of its circuitry and functionality in response to new inputs, as well as the ability of neural tissue to recover from injury by reorganizing its function to compensate for partial destruction of tissue or loss of function caused by degenerative disorders. Thus, neural plasticity refers to both increased modifiability, in the sense of being able to learn an altered circuitry in response to specific experiences, and also to increase the capacity to repair in the sense of being able to reorganize following various forms of neural damage.

In the context of the present invention, “critical stage kitten” refers to a feline when its visual cortex is at the height of a critical stage. The term “critical stage” refers to the point in neural development when the neurons are at or near the height of neural plasticity. Generally, this occurs when the kitten is between about 24-35 days old; typically, when it is between about 26-32 days old; and preferably when it is about 28 days old.

In the context of the present invention, an “adult feline” refers to a feline generally at least four months of age, preferably at least six months of age.

In another aspect of the invention, cDNA libraries are constructed from the visual cortex of a dark-reared feline using the same techniques described above. In the context of the present invention, the term “dark-reared feline” refers to felines that have been reared from before the age of eye opening (8-12 days old) to at least four months of age, with no more than 1 hour continuous exposure to light; preferably with no more than 10 minutes continuous exposure to light; and, even more preferably, these felines have never been exposed to light. These felines are characterized by delayed development of the visual cortex. In other words at four months, their cortex remains at a critical stage, exhibiting a high degree of neural plasticity.

In a preferred embodiment of the present invention, a first set of cDNA libraries are constructed from the differentially expressed sequences between the critical stage kittens and the adult felines. A second set of cDNA libraries are constructed from the differentially expressed sequences between the dark-reared felines and the adult felines. Then, using any one of several techniques, the commonly expressed sequences are extracted and form the preferred cDNA libraries. Suitable techniques include any of the techniques described above. Preferably, subtractive probes, formulated from the second cDNA libraries, are used to screen the first libraries. In the context of the present invention, these commonly expressed sequences will also be referred to as “differentially expressed sequences.”

The differentially expressed sequences comprising any one of the above cDNA libraries are verified using any one of several techniques, including Northern blot hybridization, described in detail in Sambrook et al., Molecular Cloning: A Laboratory Manual, Cold Spring Harbor Laboratory, 1989, incorporated herein by reference. The differentially expressed sequences which are generally at least two-fold enriched (based on band intensity comparisons), typically at least three-fold enriched, and preferably at least four-fold enriched are likely to be truly representative of the differentially expressed sequences. Some sequences represent mRNAs of abundance too low to be detected on the total cellular RNA blots. In these circumstances, antisense riboprobes are used to detect transcripts on the blots containing poly (A) RNA or total RNA from the first group. Alternatively, quantitative polymerase chain reaction may be used to detect or verify differences in expression.

In order to sequence any specific cDNA, it generally is isolated and purified from all the other sequences. This can be accomplished using any one of several techniques, including alkaline lysis described in detail in Sambrook et al., Molecular Cloning: A Laboratory Manual, Cold Spring Harbor Laboratory, 1989. Briefly, in one embodiment, a bacterial colony containing the cDNA of interest is identified and further amplified. Once cDNA is amplified from the mixed clone library, it can be used as a template for further procedures such as nucleotide sequencing.

The sequences, constructs, vectors, clones, and other materials comprising the present invention can advantageously be in the enriched or isolated form. Within the context of the present invention, “enriched” means that the concentration of the material is at least about 2, 3, 4, 10, 100, or 1000 times its natural concentration, for example, advantageously 0.01% by weight, preferably at least about 0.1% by weight. Enriched preparations of about 0.5%, 1%, 5%, 10%, and 20%, by weight, are also contemplated.

Within the context of the present invention, the term “isolated” requires that the material be removed from its original environment (e.g., the natural environment if it is naturally occurring). For example, a naturally occurring polynucleotide present in a living animal is not isolated, but the same polynucleotide, separated from some or all of the coexisting materials in the natural system, is isolated.

It is also advantageous that the sequences be in purified form. Within the context of the present invention, the term “purified” does not require absolute purity; rather, it is intended as a relative definition. The cDNA clones are not naturally occurring as such, but rather are obtained via manipulation of a partially purified naturally occurring substance (messenger RNA) The conversion of mRNA into a cDNA library involves the creation of a synthetic substance (cDNA) and pure individual cDNA clones can be isolated by screening the library with specific probes. Thus, creating a cDNA library from messenger RNA and subsequently isolating individual clones from that library results in a single clone in substantially pure form.

One of ordinary skill in the art will appreciate that the human polynucleotides may be identified and isolated given the feline sequences. Human sequences share at least 80% homology with feline sequences. When the comparison is extended to the amino acid level, sequence identity is increased to 92%. Once the corresponding human sequences have been identified and isolated, the associated polypeptides may be identified and produced.

Polynucleotides of the present invention include the polynucleotides depicted by the specific sequences set forth in the Sequence Listing and designated SEQ ID NO: 1-SEQ ID NO: 132. In. the context of the present invention, the term “polynucleotide” is intended to refer to the polynucleotides represented by SEQ. ID. NOS.: 1-132, complementary sequences thereto, allelic variations thereof, or portions thereof, and cDNA clones related thereto. These polynucleotides are also included in the differentially expressed sequences described in detail above.

In addition to the claims to individual polynucleotides, it is intended that the present disclosure also supports claims to numerical subgroupings. Thus, subgroupings of 50 polynucleotides, and their corresponding sequences, are contemplated as being within the scope of this invention, as are subgroupings of 5, 10, 25, and 100 polynucleotides.

Knowledge of the sequences of the polynucleotide of the present invention permits routine isolation and sequencing of the complete coding sequence of the corresponding gene. The complete coding sequence is present in a full-length cDNA clone as well as in the gene coding region on the genomic clones. Therefore, each partial polynucleotide of the present invention corresponds to a cDNA from which it was derived, a complete genomic gene sequence, a polypeptide coding region, and a polypeptide or amino acid sequence encoded by that region.

The polynucleotides of the present invention are generally of sufficient length to effect preliminary identification and exact chromosome mapping. Accordingly, the polynucleotides disclosed herein are generally at least 50 base pairs in length, typically in the range of about 90 to 500 base pairs in length, and preferably in the range of about 150 to 500 base pairs in length. The length is ultimately determined by the quality of sequencing data and the length of the cloned cDNA.

Raw data from automated sequencers and manual sequencing efforts are edited to remove low quality sequences at the end of the sequence run. High quality sequences, usually a result of sequencing templates without excessive salt contamination, generally give about 400 base pairs of reliable sequence data; shorter sequences give fewer bases of reliable data. A 50-base pair polynucleotides is long enough to be translated into about a 16 amino acid peptide sequence. This length is sufficient to observe similarities when they exist in a database search. Also, 50 nucleotides are generally sufficient for unique identification of specific location in genomic DNA of a sequence coding for unique protein. Furthermore, a 50-base pair sequence is long enough to design a PCR primer from the sequence to amplify the complete polynucleotides.

The polynucleotides of the present invention are highly specific markers or probes for the corresponding complete gene coding regions and complete genes conferring neural plasticity. The polynucleotides of the present invention are partial sequences, in other words, they represent a relatively small coding region or untranslated region of the genes. However, the disclosed polynucleotides will hybridize, under sufficiently stringent conditions, only with that gene to which they correspond in any species, i.e., feline and human. Suitably stringent hybridization conditions include, for example, a sequence identity of at least about 80% base pair identity and, preferably, at least 90% base pair identity. This property permits the use of the polynucleotides of the present invention to isolate the entire coding region and even the entire sequence using any one of several suitable techniques, including 5′-RACE Polymerase Chain Reaction, described in detail in Innis et al., PCR Protocols: A Guide to Methods and Applications, Academic Press, Inc., 1990, incorporated herein by reference. Therefore, only routine laboratory work is necessary to parlay the unique sequence into the corresponding unique complete gene sequence.

The first step in determining where a polynucleotide is located in the genomic region is to analyze the sequence for the presence of a coding sequence. This can be accomplished using any one of several suitable means, including the CRM program, which predicts the extent and orientation of the coding region of a sequence or the ESEE program Cabot and Beckenbach, “Simultaneous editing of multiple nucleic acids and protein sequences with ESEE,” Comput. Applic. Biosci. 5:233-234, 1989, incorporated herein by reference. Based on this information, one can infer the presence of start or stop codons within a sequence and whether the sequence is coding or not. If stop or start codons are present, then the polynucleotide can cover both parts of the 5′-untranslated or the 3′-untranslated part of the mRNA as well as part of the coding sequence. If no coding sequence is present, it is likely that the differentially expressed molecule is derived from the 3′-untranslated sequence due to its longer length and the fact that most cDNA library construction methods are biased with the 3′ end of the mRNA. Based on this information, complete sequences can be obtained from polynucleotides of the present invention by any suitable means known in the art, including 5′-RACE polymerase chain reaction and cDNA screening. The polynucleotides of the present invention are specific tags for a messenger RNA molecule. The complete sequence of that messenger RNA, in the form of cDNA, can be determined using the polynucleotide as a probe to identify a cDNA clone corresponding to a full-length transcript followed by sequencing of that clone. The polynucleotide or the full-length cDNA clone can also be used as a probe to identify a genomic clone or clones that contain the complete gene including regulatory and promoter regions, exons, and introns.

Polynucleotides of the present invention can be used as probes to identify the full length cDNA clones from which they were derived or to screen other cDNA libraries. Corresponding cDNA clones display at least a 90% homology, typically a 95% homology, and preferably a 97% homology to the coding region of the polynucleotides of the present invention.

One of ordinary skill in the art will appreciate that the probes may be made and used employing any one of several techniques, including, for example, nick translating and random primer labeling with ³²P using DNA polymerases, described in detail in Sambrook et al., Molecular Cloning: A Laboratory Manual, Cold Spring Harbor Laboratory, 1989, incorporated herein by reference. A lambda library can then be directly screened with the labeled sequences of interest or the library can be converted en masse to pBluescript (Stratagene, La Jolla, Calif.) to facilitate bacterial colony screening using methods known in the art. Briefly, filters with bacterial colonies containing the library of pBluescript or bacterial lines containing lambda plaques are denatured and the DNA is fixed to the filters. The filters are hybridized with the labeled probe, using hybridization conditions described by Davis et al., Basic Methods in Molecular Biology, Elsevier Press, NY, 1986, incorporated herein by reference. The polynucleotides of the present invention, cloned into lambda or pBluescript, can be used as positive controls to assess background findings and to adjust the hybridization and washing stringencies necessary for accurate clone identification. The resulting autoradiograms are compared to duplicate plates of colonies or plaques; each exposed spot corresponds to positive colony or oplaque. The colonies or plaques are selected, expanded, and the DNA is isolated from the colonies for further analysis and sequencing.

Positive cDNA clones in phage lambda are analyzed to determine the amount of additional sequence they contain using PCR with one primer from the polynucleotides of the present invention and the other primer from the vector. Clones with a larger insert PCR product than the original differentially expressed molecule clone are analyzed by restriction digestion and DNA sequencing to determine whether they contain an insert of the same size or similar size as the mRNA on a Northern blot.

Once one or more overlapping cDNA clones are identified, the complete sequence of the clones can be determined. The preferred method is to use exonuclease III digestion, McCombie et al., Methods 3:33-40, 1991, incorporated herein by reference. Briefly, a series of deletion clones is generated, each of which is sequenced. The resulting overlapping sequences are assembled into a single contiguous sequence of high redundancy (usually 3-5 overlapping sequences at each nucleotide position), resulting in a highly accurate final sequence.

A similar screening and clone selection approach can be applied to obtaining cosmid or lambda clones from a genomic DNA library that contains the complete gene from which the polynucleotide was derived (Kirkness et al., Genomics 10:985-995, 1991). Although the process is much more laborious, these genomic clones can be sequenced in their entirety also. A shotgun approach is preferred to sequencing clones with inserts longer than 10 kb (genomic cosmid and lambda clones). In shotgun sequencing, the clone is randomly broken into many small pieces, each of which is partially sequenced. The sequence fragments are then aligned to produce the final contiguous sequence with high redundancy. An intermediate approach is to sequence just the promoter region and the intron-exon boundaries and to estimate the size of the introns by restriction endonuclease digestion.

Using the sequence information provided herein, the polynucleotides of the present invention can be derived from natural sources or synthesized using known methods. Sequences falling within the scope of the present invention are not limited to the specific sequences described, but include the corresponding human sequences, complementary sequences, allelic and species variations thereof, and portions thereof of at least 15 to 18 base pairs. (Sequences of at least 15 to 18 bases can be used, for example, as PCR primers or as DNA probes.) Preferably, sequences are at least 50 base pairs in length.

In addition, the invention includes the entire coding sequence associated with the specific polynucleotides of bases described in the sequence listing, as well as portions of the entire coding sequence of at least 15-18 bases and allelic and species variations thereof. Furthermore, to accommodate codon variability, the invention includes sequences coding for the same amino acid sequences as do the specific sequences disclosed herein. Finally, although the error rate in the manual and automated sequencing techniques used in the present invention is small, there remains some chance of error. Therefore, claims to particular sequences should not be so narrowly construed as to require inclusion of erroneously identified bases or exclude corrections.

Any specific sequence disclosed herein can be readily screened for errors by resequencing in both directions (i.e., sequence both strands of cDNA).

In another aspect of the invention, the invention relates to those sequences of SEQ. ID. NOS.: 93-120 that comprise the cDNA coding sequence for known polypeptides as depicted in Table I.

TABLE I

| Seq. ID | Clone | Putative | | | | % | Length |
|---|---|---|---|---|---|---|---|
| No. | No. | Identification | Species | Accession | SCORE | ID | (nt/aa) |
| 120 | pKVC4 | alpha internexin | Rat | RNINTLAA | 251 | 72 | 169 nt |
| 119 | pKVC6 | TAPA-1 | Human | HSTAPA1 | 745 | 87 | 244 nt |
| 118 | pKVC6B | Amphiphysin | Chicken | GDAMPHIP | 113 | 47 | 38 aa |
| 117 | pKVC9 | v-fos | Human | HSFTE1A | 975 | 93 | 277 nt |
| | | transformation | | | | | |
| | | effector | | | | | |
| 116 | pKVC10 | Cytoskeletal gamma | Human | HSACTCGR | 245 | 84 | 88 nt |
| | | actin | | | | | |
| 115 | pKVC12 | 90 kD heat shock | Human | HSHSP90R | 320 | 76 | 161 nt |
| | | protein | | | | | |
| 114 | pKVC17 | Substance P | Rat | RNSPR05 | 214 | 96 | 57 nt |
| | | receptor | | | | | |
| 113 | pKVC18 | Cytochrome oxidase | Human | MIHSCG | 686 | 80 | 266 nt |
| | | I | | | | | |
| 112 | pKVC21 | nrp-lb | Xenopus | XLNRP | 129 | 57 | 200 nt |
| 110 | pKVC22 | clq beta isoform | Human | HSC1QBR | 221 | 94 | 47 aa |
| 109 | pKVC27 | Cytopolasmic | Rat | RNGLTA | 820 | 36 | 272 nt |
| | | Succinyl CoA | | | | | |
| | | synthetase | | | | | |
| 108 | pKVC35 | Vacuolar proton | Human | HSPCHSUC | 575 | 80 | 250 nt |
| | | ATPase channel | | A | | | |
| 107 | pKVC46 | 71 kD heat shock | Human | HSHSC70 | 582 | 83 | 240 nt |
| | | cognate protein | | | | | |
| 105 | pKVC49 | Contactin | Chicken | GGCONTAC | 330 | 69 | 223 nt |
| 104 | pKVC55 | LAMP-1 | Human | HSLAMP1A | 619 | 77 | 293 nt |
| 102 | pKVC79 | Nuclear encoded | Bovine | BTADTPMT | 377 | 98 | 74 aa |
| | | ADP/ATP transporter | | | | | |
| 101 | pKVC80 | VAMP-2 | Rat | RNVAMPB | 461 | 76 | 253 nt |
| 100 | pKVC82 | Carboxypeptidase E | Human | HSCARBE | 558 | 83 | 203 nt |
| 99 | pKVC86 | Ribosomal protein | Rat | RRS27 | 300 | 76 | 132 nt |
| | | S27 | | | | | |
| 98 | pKVC90 | hnRNP core protein | Human | HSRNPA1 | 880 | 94 | 248 nt |
| | | A1 | | | | | |
| 97 | pKVC91 | NADH dehydrogenase | Bovine | MIBTXX | 206 | 79 | 51 aa |
| 96 | pKVC92 | Mitochondrial hinge | Human | HSHINGE | 214 | 64 | 135 nt |
| | | protein | | | | | |
| 95 | pKVC105 | mRNA for prolif. | Human | HSPAG | 98 | 77 | 48 aa |
| | | associated protein | | | | | |
| 94 | pKVC108 | Initiation factor | Mouse | MMEIF4AL | 445 | 75 | 254 nt |
| | | eIF-4A | | | | | |
| 93 | pKVC134 | alpha tubulin | Human | HSHA44G | 312 | 100 | 60 aa |

These sequences have greater than 90% identity with known amino acid sequences.

Another aspect of the present invention relates to those polynucleotides encoded by sequences designated as one of SEQ. ID. NOS.: 1-92 and 121-132. These sequences encode polypeptides having little or no similarity to known amino acid sequences. These polynucleotides can be parlayed into their associated polypeptides using any one of a variety of techniques, including, for example, synthesizing the polypeptide encoded by the polynucleotide using commercially available peptide synthesizers including the Applied Biosystems Peptide Synthesizer (Perkin-Elmer). This is particularly useful in producing small peptide end fragments of larger polypeptides. Generally, the term “associated polypeptides” includes polypeptide demonstrating an identity of greater than 80%, typically greater than 90%, and preferably greater than 92% to the polynucleotides of the present invention.

Alternatively, the associated polypeptides may be produced by inserting the polynucleotides into a host organism capable of expressing the polynucleotide. Suitable organisms include bacteria, yeast, a cell line, or a multicellular plant or animal. The literature is replete with examples of suitable host organisms and expression techniques. For example, naked polynucleotide (DNA or mRNA) can be injected directly into muscle tissue of animals where it is then expressed. Alternatively, the coding sequence, together with appropriate regulatory regions (i.e., a construct) can be inserted into a vector, which is then used to transfect or infect a cell. The cell, which may or may not be part of a larger organism, then expresses the polypeptide.

Recombinant binding partners of the present invention include proteins (e.g., antibodies), peptides and small organic molecules. Antibodies generated against the polypeptide corresponding to a sequence of the present invention can be obtained by direct injection of the naked polynucleotide into an animal or by administration of the polypeptide to an animal, preferably a non-human, using methods known in the art. The antibody so obtained will then bind the polypeptide itself. In this manner, even a sequence encoding only a fragment of the polypeptide can be used to generate antibodies binding the whole native polypeptide. Such antibodies can then be used to isolate the polypeptide from tissues expressing that polypeptide. Moreover, a panel of such antibodies specific to a large number of polypeptides, can be used to identify and differentiate such tissue.

Despite the potential utility of antibodies as recombinant binding partners, there may be pharmaceutical applications for which they are not appropriate due to their cost, potential for immunogenicity, or need for specialized forms of delivery such as orthotopic or oral administration. For these purposes, small organic compounds or peptides may also be developed. Such peptides and compounds may be developed through: (1) screening of bacterial peptide expression libraries, antibody paratope analogs or antibody Fab expression libraries to identify peptide or antibody variable region inhibitors (Gene 73:305, 1988; Proc. Nat. Acad. Sci. USA 87:6378, 1990; BioChromatography 5:22, 1990; Protein Engineering 3:641, 1989); (2) rational drug design programs using antibodies as a “pharmacophore” to create organic molecule analogs (Biotechnology, Jan. 19, 1991), or traditional rational drug design programs using crystallized vitamin receptor to identify peptide or organic inhibitors (Biochem. J. 268:249, 1990; Science 248:1544, 1990); and (3) screening a library of organic molecules.

Other assays can also prove useful, including specific binding assays using antibodies which act as competitive antagonists. Through these means a repertoire of protein and non-protein molecules suitable for human use can be generated, and may be used to define optimal regimens for antagonizing or upregulating the activity of polypeptides encoded by the polynucleotides.

The amount of recombinant binding partners and timing of administration is determined by in vitro study followed by in vivo experimentation.

Another aspect of the present invention includes constructs including one or more of the polynucleotides, as broadly described above. The constructs comprise a vector, such as a plasmid or viral vector, into which a polynucleotide of the present invention has been inserted, in either a sense or antisense orientation. Preferably, the construct further contains regulatory regions, including, for example, a promoter, operably linked to the polynucleotide. Large numbers of suitable vectors and promoters are known and are commercially available. The following vector constructs are provided by way of example:

Bacterial: pBs, phase script OX174, pBluescript SK, pBsKs, pNH8a, pNH16a, pNH1a, pNH46a (Stratagene), ptrc99A, pKK223-3, pKK233-3, pDR540, pRIT5 (Pharmacia).

Eukaryotic: pWLneo, pSV2cat, pOG44, pXT1, pSG (Stratagene), pSVK3, pBPV, pMSG, pSVL (Pharmacia).

Viral: HSV-1, retroviral, adenoviral and vaccinia virus. See, for example, U.S. patent application Ser. No. 08/213,799 incorporated herein by referrence.

Promoter regions can be selected from any desired gene using chloramphenicol transferase (“CAT”) vectors or other vectors with selectable markers. Two appropriate vectors are pKK232-8 and pCM7. Particular named bacterial promoters include lacI, lacZ, T3, T7, gpt, lambda P_{RO} and trc. Eukaryotic promoters include CMV immediate early, HSV thymidine kinase, early and late SV40, LTRs from retrovirus and mouse metallothionein-I. Selection of the appropriate vector and promoter is well within the level of ordinary skill in the art.

In a further embodiment, the present invention relates to host cells containing the above-described construct. The host cell can be a higher eukaryotic cell, such as a mammalian cell; or a lower eukaryotic cell, such as a yeast cell; a prokaryotic cell, such as a bacterial cell. Introduction of the construct into the host cell can be affected using any one of several methods known in the art, including by calcium phosphate transfection, DEAE dextran mediated transfection, infection, or electroporation, as described in detail in Davis et al., Basic Methods of Molecular Biology, 1986, incorporated herein by reference.

The constructs in host cells can be used in a conventional manner to produce the gene product coded by the recombinant sequences as described above, or the host cells can be administered directly to an animal in need thereof as described below. Alternatively, the encoded polypeptide can be synthetically produced by conventional peptide synthesizers.

The polynucleotides of the present invention generally possess the capability of encoding polypeptides associated with energy metabolism and mitochondrial function. These peptides are associated with the special needs of the critical period which requires high levels of energy to be expended within the CNS. The polynucleotides of the present invention play an important role in providing the necessary elevated function and increased energy metabolism associated with this process.

The polynucleotides of the present invention generally possess the capability of encoding polypeptides associated with cell membrane associated proteins. Cell membrane associated proteins may be neurotransmitter receptors. As such, they may increase the sensitivity of neurons to various neurotransmitters and hormones. This results in the activation of intercellular cascades, increasing neural plasticity.

The polynucleotides of the present invention generally possess the capability of encoding polypeptides associated with neurotransmitter release and processing associated proteins. Thus, the polynucleotides of the present invention administered to a warm-blooded animal would increase neurotransmitter releases, altering the strength of connections between the cells. The change in neurotransmitter release and increase synthesis of neurotransmitter represents a prime example of increased neural plasticity.

The polynucleotides of the present invention generally possess the capability of encoding polypeptides associated with cell or tissue remodeling associated proteins. These proteins include heat shock proteins which are known to play a role in protecting cells in the body from various stressers. Up-regulation of the function of this protein would assist in neural protection.

The polynucleotides of the present invention generally possess the capability of encoding polypeptides associated with cytoskeletal proteins. These polypeptides control rigidity and direction of the growth of neurons during their regenerative and plastic phases. The cytoskeletal proteins associated with the polynucleotides of the present invention play a role in directing cell and fiber growth during the recovery of function after neural injury. The polynucleotides of the present invention generally possess the capability of encoding polypeptides associated with mRNA transcription and processing. Thus, these sequences play a role in facilitating the differentials transcription of the described sequences.

The polynucleotides and complete gene sequences of the present invention are also valuable for chromosome identification. Each sequence is specifically targeted to, and can hybridize with, a particular location on an individual chromosome. Moreover, there is a current need for identifying particular sites on the chromosome. The present invention constitutes an expansion of the available chromosome markers. Using any one of several techniques, the polynucleotides and the corresponding complete sequences can similarly be mapped to chromosomes. The mapping of the polynucleotides to chromosomes according to the present invention is an important first step in correlating those sequences with genes associated with genetic disorders.

Briefly, within one embodiment, sequences can be mapped to chromosomes by preparing PCR primers (preferably 15-25 base pairs) from the polynucleotides of the present invention. Computer analysis of the polynucleotide is used to rapidly select the primers that do not span more than one exon in the genomic DNA, thus complicating the amplification process. These primers are then used for PCR screening of somatic cell hybrids containing individual human chromosomes. Only those hybrids containing the human gene corresponding to the polynucleotides of the present invention will yield an amplified fragment.

PCR mapping of somatic cell hybrids is a rapid procedure for assigning a particular differentially expressed molecule to a particular chromosome. Three or more clones can be assigned per day using a single thermal cycler. Using the present invention with the same oligonucleotide primers, sublocalization can be achieved with panels of fragments from specific chromosomes or pools of large genomic clones in analogous manner. Other mapping strategies that can similarly be used to map a polynucleotide to its chromosome include in situ hybridization, prescreening with labeled flow sorted chromosomes and preselection by hybridization to construct chromosome specific cDNA libraries.

Fluorescence in situ hybridization (FISH) of a cDNA clone to a metaphase chromosomal spread can be used to provide a precise chromosomal location. This technique can be used with cDNA as short as 500 or 600 bases; however, clones larger than 2000 base pairs have a higher likelihood of binding to a unique chromosomal location with sufficient signal intensity for simple detection. FISH requires the use of the clone from which the polynucleotide was derived, and the longer the better. Two thousand base pairs is good, 4,000 base pairs is better, and more than 4,000 is probably not necessary to get results in a reasonable percentage of the time. For a review of this technique, see Verma et al., Human Chromosomes: A Manual of Basic Techniques, Pergamon Press, New York, 1988, incorporated herein by reference.

Reagents for chromosome mapping can be used individually to mark a single chromosome or a single site on that chromosome or as panels of reagents, for marking multiple sites and/or multiple chromosomes. Reagents corresponding to noncoding regions of the genes actually are preferred for mapping purposes. Coding sequences are more likely to be conserved within gene families, thus increasing the chance of cross-hybridization during chromosomal mapping.

Once a sequence has been mapped to a precise chromosomal location, the physical position of the sequence on the chromosome can be correlated with genetic map data. The relationship between genes and diseases that have been mapped to the same chromosomal region are then identified through linkage analysis, coinheritance of physically adjacent genes. See, for example, V. McKusick, Mendelian Inheritance in Man, available on line through Johns Hopkins University Welch Medical Library, incorporated herein by reference.

Next, it is necessary to determine the differences in the cDNA or genomic sequence between affected and unaffected individuals. If a mutation is observed in some or all of the affected individuals but not in any normal individuals, then the mutation is likely to be causative agent of the disease.

With current resolution of physical mapping and genetic mapping techniques, a cDNA precisely localized to a chromosomal region associated with the disease could be one of between 50 and 500 potential causative genes. This assumes one megabase mapping resolution and one gene per 200 kilobases.

Comparison of affected and unaffected individuals generally involves first observing structural alterations in the chromosomes, such as deletions or translocations that are visible from chromosomal spreads or detectable using PCR based on that cDNA sequence. Ultimately, complete sequencing of genes from several individuals is required to confirm the presence of a mutation and to distinguish mutations from polymorphisms.

In addition to the foregoing, the sequences of the invention, as broadly described, can be used to control gene expression through triple helix formation or antisense DNA or RNA, both of which methods are based on binding of a polynucleotlde to DNA or RNA. Polynucleotides suitable for use in these methods are usually 20 to 40 bases in length and are designed to be complementary to a region of the gene involved in transcription (triple helix; see Lee et al., Nucl. Acids Res. 6:3073, 1979; Cooney et al., Science 241:456, 1988; and Dervan et al., Science 251:1360, 1991) or to mRNA itself (Antisense-Okano, J. Neurochem. 56:560, 1991; Oligodeoxynucleotides as Antisense Inhibitors of gene Expression, CRC Press, Boca Raton, Fla., 1988). Triple helix formation optimally results in a shutoff of RNA transcription from DNA, while antisense RNA hybridization blocks translation of an mRNA molecule into polypeptide. Information contained in the sequences of the present invention is necessary and sufficient for the design of an antisense or triple helix oligonucleotide.

The polynucleotides of the present invention are useful tools for the treatment of degenerative disorders, for assisting neural cell repair and regrowth, the treatment of learning disorders, and for enhanced memory and learning capacity using gene therapy.

Those polynucleotide sequences that are associated with neural plasticity may be empirically tested in vivo, by formulating the antisense construct or the triple helix construct or a polynucleotide of the present invention and inserting the sequence into the visual cortex of a critical stage kitten using any means described above. Those sequences which are capable of blocking neural plasticity in critical stage kittens will be therapeutically effective. The result can be subsequently assayed using any one of a variety of techniques, including electrophysiological methods described in Kandel et al., Principles of Neural Science, 3d ed., Elsevier Press, NY, 1991, incorporated herein by reference, and, alternatively, by anatomical measures of lateral geniculate cell size, which would normally show shrinkage of cells associated with visual deprivation. Those areas in which the constructs have been effused used would exhibit no shrinkage.

In one aspect of the invention the polynucleotides of the present invention are administered to treat degenerative disorders. In the context of the present invention, the term “degenerative,” as applied to disorders of the nervous system, is used to designate a group of disorders in which there is a gradual, generally symmetric, relentlessly progressive wasting away of neurons. The term “degenerative diseases of the nervous system” is intended to include any of the diseases referred to in Table II, as well as other brain disturbances including, but not limited to, depression, dementia, and schizophrenia. This term is used interchangeably with the terms “diseases with a neurological dysfunction or disorder” or “neurodegenerative diseases,” which are intended to have the same meaning.

Since etiological classification of such disorders is virtually impossible, subdivision of degenerative diseases into individual syndromes rests on descriptive criteria, based largely on pathologic anatomy but to some extent on clinical aspects as well. Table II groups the disease states according to the outstanding clinical features that may be found in an actual case.

Table II
CLINICAL CLASSIFICATION OF THE DEGENERATIVE
DISEASES OF THE NERVOUS SYSTEM

| | |
|---|---|
| I. | Syndrome in which progressive dementia is |
| | an outstanding feature in the absence of |
| | other prominent neurologic signs |
| | A. Diffuse cerebral atrophy |

1. Senile dementia
2. Alzheimer's disease

| | B. Circumscribed cerebral atrophy (Pick's |
|---|---|
| | disease) |
| II. | Syndrome in which progressive dementia is |
| | combined with other neurologic signs |
| | A. Principally in adults |

1. Huntington's chorea
2. Cerebrocerebellar degeneration

B. In children and adults

```
1. Amaurotic family idiocy (neuronal
  lipidoses)
2. Leukodystrophy
3. Familial myoclonus epilepsy
4. Hallervorden-Spatz disease
5. Wilson's disease
  (hepatolenticular degeneration,
  Westphal-Strumpell
  pseudosclerosis)
```

| | |
|---|---|
| III. | Syndrome chiefly manifested by gradual |
| | development of abnormalities of posture or |
| | involuntary movements |
| | A. Paralysis agitans |
| | B. Dystonia musculorum deformans (torsion |
| | dystonia) |
| | C. Hallervorden-Spatz disease and other |
| | restricted dyskinesias |
| | D. Familial tremor |
| | E. Spasmodic torticollis |
| IV. | Syndrome chiefly manifested by slowly |
| | developing ataxia |
| | A. Cerebellar degenerations |
| | B. Spinocerebellar degenerations |
| | (Friedrich's ataxia, Marie's |
| | hereditary ataxia) |
| V. | Syndrome with slowly developing muscular |
| | weakness and wasting |
| | A. Without sensory changes; motor system |
| | disease |

```
1. In adults
  a. Amyotrophic lateral
    sclerosis
  b. Progressive muscular atrophy
  c. Progressive bulbar palsy
  d. Primary lateral sclerosis
2. In children or young adults
  a. Infantile muscular atrophy
    (Werdnig-Hoffmann disease)
  b. Other rows of familial
    progressive muscular atrophy
    (including Wohlfart-
    Kugelberg-Welander syndrome)
  c. Hereditary spastic
    paraplegia
```

B. With sensory changes

```
1. Progressive neural muscular
  atrophy
  a. Peroneal muscular atrophy
    (Charcot-Marie-Tooth)
  b. Hypertrophic interstitial
    neuropathy (Dejerine-Sottas)
2. Miscellaneous forms of chronic
  progressive neuropathy
```

| | |
|---|---|
| VI. | Syndrome chiefly manifested by progressive |
| | visual loss |
| | A. Hereditary optic atrophy (Leber's |
| | disease) |
| | B. Pigmentary degeneration of the retina |
| | (retinitis pigmentosa) |

In one embodiment of the present invention, the polynucleotides of the present invention are inserted into a construct, as described above, or delivered by other means known in the art and administered to an animal suffering from, or genetically susceptible to, a neurodegenerative disease or diseases of tissue which share a common embryological basis with the nervous system. This results in the amelioration of the primary neurological symptoms of the neurodegenerative disease. Similar improvement in overall functional ability should also be seen.

In another aspect of the present invention, the polynucleotides are administered to assist the recovery of injured neurons. Neurons can be injured as a result of a direct injury or disease. For example, acute injuries to the central nervous system or peripheral nervous tissue can occur from, among others, stroke, brain injury or spinal cord injury. The insertion of the polynucleotides of the present invention confers the ability to direct growing or regenerating axons within the central nervous system and peripheral nervous system and enabling them to form synapses with their neighbors.

In another aspect of the present invention, the polynucleotides are used to treat learning disorders. In the context of the present invention, the term “learning disorder” refers to disorders which are characterized by a decreased ability to process and store information. Such learning disorders include dyslexia, dysphoria, aphasia, disgraphia, mental retardation, including Down's Syndrome. Administration, as described in detail below, of the polynucleotides, as broadly described above, results in improvement in overall functional ability.

In another aspect of the present invention, the polynucleotides are used to treat those who do not indicate obvious pathology. Normal individuals so as to enhance memory and learning capacity. In the context of the present invention, the term “enhanced memory and learning capacity” refers to the increased capacity to process and store information. Administration of the polynucleotides, as broadly described above, should result in increased learning capacity.

Using methods known in the art and described in detail below, the polynucleotides are delivered to the particular neuronal tissue in need. At the new location the genes provide neural plasticity; essentially providing the cell with the necessary material to compensate for the degeneration. Preferably, the nucleotides are administered in a construct, however, they may be delivered by any suitable means known in the art.

The constructs can be administered by any means which will ensure that it reaches the desired location. Preferably, the constructs are directed to the portion affected, however, general administration would not have an adverse effect. The specific regions of the brain are reviewed in Mayeux, E. and Kandel, E. R., Chapter 54 (pp. 839-851) in Kandel et al., Principles of Neural Science, 3d ed., Elsevier Press, NY, 1991, incorporated herein by reference.

One of the biggest impediments to delivery of pharmaceuticals to the central nervous system is the blood-brain barrier. In the context of the present invention, the term “blood-brain barrier” refers to the blood-brain barrier made up of brain microvessel endothelial cells characterized by tight intercellular junctions, minimal pinocytic activity, and the absence of fenestra. These characteristics endow these cells with the ability to restrict passage of most small polar blood-borne molecules (e.g., neurotransmitters, including catecholamines and neuropeptides) and macromolecules (e.g., proteins) from the cerebrovascular circulation to the brain. The blood-brain barrier contains highly active enzyme systems as well, which further enhance the already very effective protective function. It is recognized that transport of molecules to the brain is not determined solely by the molecular size but by the permeabilities governed by the specific chemical characteristics of the permeating substance. Thus, besides molecular size and lipidicity, the affinity of the substances to various blood proteins, specific enzymes in the blood, or the blood-brain barrier, will considerably influence the amount of the drug reaching the brain. Several mechanisms for crossing the blood-brain barrier are described below and others are known in the art.

The term “treatment” as used within the context of the present invention, refers to reducing or alleviating symptoms in a subject, preventing symptoms from worsening or progressing, inhibition or elimination of the causative agent, or prevention of the infection or disorder in a subject who is free therefrom. Thus, for example, treatment of infection includes destruction of the infecting agent, inhibition of or interference with its growth or maturation, neutralization of its pathological effects and the like. A disorder is “treated” by partially or wholly remedying the deficiency which causes the deficiency or which makes it more severe. An unbalanced state disorder is “treated” by partially or wholly remedying the imbalance which causes the disorder or which makes it more severe.

There are unique considerations in the treatment of central nervous system dysfunction. Unlike other tissues, brain tissue is not redundant. It is highly differentiated, compartmentalized, and irreplaceable. Thus neuropharmaceutics must be found non-toxic to normal tissues. However, it has been difficult to find the most efficacious route circumventing the blood-brain barrier.

One way to bypass the barrier is by intracerebral spinal fluid administration by lumbar puncture or by the intraventricular route. Catheterization using the aommaya reservoir is used, but logistics dictate that to be a last resort.

Because the barrier is selective, some drugs can be administered orally. Since lipophilac chemicals or agents that mimic the neural amino acids can bypass the barrier by mere diffusion or by transport via the energy-dependent membrane bound character, respectively. Thus constructs can be prepared to add lipid and/or carbohydrate groups to the construct to make it more lipophilic and then hence the ability to cross the blood-brain barrier.

Transient reversible modification of the blood-brain barrier is accomplished in either of two ways—osmotic opening or metrozol opening. The first method is based on increasing capillary permeability by osmotically induced shrinkage of the endothelial cells which cause the widening of the intercellular-type junctions. The osmotic load is generally a hyperosmotic water-soluble agent such as mannitol or arabinose. Briefly, under general anesthesia, a transfemoral catheter is introduced into the internal carotid or vertebral artery and 150-300 ml infusion of 25% mannitol is administered at 6-10 mg/sec for thirty seconds. The intravenous infusion of the construct of the present invention is begun approximately 5-7 minutes before the mannitol infusion and is continued for fifteen minutes. The transfemoral catheter is removed and the patient observed for 24-48 hours.

Alternatively, the active agent (the construct or polynucleotide) may be linked to the osmotic agent (mannitol, arabinose, glucose or other sugar moiety), and a single infusion may be used. Conventional techniques may be used to link the active agent and the osmotic agent. The linked agent itself will then cause the osmotically induced shrinkage of the endothelial cells in order to widen the tight intracellular junctions. The linked agent may be designed such that the active agent is cleared from the linked agent after the blood-brain barrier has been crossed.

In the second method, capillary permeability is increased by eliciting seizure activity using a central nervous system stimulant such as pentylenetetrazol. This technique is similar to that of osmotic opening with replacement of mannitol infusion by parenteral delivery of the stimulant.

A further alternative method of delivering polynucleotides to target areas of the brain is transport the polynucleotide into the brain by means of defective herpes simplex virus I (HSV I) vector using a method described by Geller et al., Science 241:1667, 1988. Particularly the defective HSV I vector described by Geller et al., supra, is pHSVlac, which contains the E. coli LacZ gene under the control of the HSV immediate early ⅘ promoter.

In order to use this HSV I vector in the present invention, the polynucleotide sequence is inserted into the defective HSV I vector using conventional techni p 7×using c This new vector containing the polynucleotide sequence can then enter the brain.

The preferred method of administration is microinjection of the polynucleotide, alone or in a pharmaceutically suitable carrier or diluent, through a stereotactically-located pipette or syringe. Suitable locations vary with application, but include intraocular and brain injections.

Pharmaceutical compositions containing the constructs in an admixture with a pharmaceutical carrier or diluent can be prepared according to conventional pharmaceutical compounding techniques. The carrier may take a wide variety of forms depending on the form of preparation desired for administration, e.g., intravenous, oral topical, aerosol, suppository, parenteral or spinal injection.

In preparing the compositions in oral dosage form, any of the usual pharmaceutical medium can be employed such as, for example, water, glycose, oils, alcohols, flavoring agents, preservatives, coloring agents, and the like.

In the case of oral liquid preparations (such as, for example, suspensions, elixirs and solutions); or carriers such as starches, sugars, diluents, granulating agents, lubricants, binders, disintegrating agents and the like.

In the case of oral solid preparations, for example, powders, capsules, and tablets. Because of their ease in administration, tablets and capsules present the most advantageous oral dosage unit form, in which solid pharmaceutical carriers are obviously employed. If desired, tablets may be sugar-coated or enteric-coated by standard techniques.

In the case of parenterals, the carrier will usually comprise sterile water, though their ingredients, for example, to aid solubility or for preservative purposes may be included. Injectable suspensions may also be prepared in which case appropriate liquid carriers such as suspending agents, pH adjusting agents, isotonicity adjusting agents and the like may be employed.

In the case of topical administration, the carrier may take a wide variety of forms, depending on the form of the preparation, such as creams, dressings, gels, lotions, ointments, or liquids.

Aerosols are prepared by dissolving or suspending the active ingredient in a propellant such as ethyl alcohol or in a propellant and solvent phase. Suppositories are prepared by mixing the active ingredient with a lipid vehicle such as theobroma oil, cacao butter, glycerin, gelatin, or polyoxyethylene glycols.

If necessary, the pharmaceutical preparations can be subjected to conventional pharmaceutical adjuvants such as preserving agents, stabilizing agents, wetting agents, salts for varying the osmotic pressure, and the like. The present pharmaceutical preparations may also contain other therapeutically valuable substances.

In another aspect of the present invention, constructs, including polynucleotides, may be delivered by chronic infusion using any suitable method known in the art, including an osmotic minipump (Alza Corp.) or delivery through a time release or sustained release medium. Suitable time release or sustained release systems include any methods known in the art, including media such as Elvax (or see, for example, U.S. Pat. Nos. 5,015,479, 4,088,798, 4,178,361, and 4,145,408). When using chronic infusion, time release, or sustained release mechanisms, the construct composition may be injected into the cerebrospinal fluid via intrathecal or intraventricular injections, as well as into the brain substances and intraocular locations.

When the gene is transfected or infected into a mammalian host cell, the mammalian cells may be administered to the patient in need thereof by any method known in the art, including that outlined in U.S. Pat. No. 5,082,670 and incorporated herein by reference.

The polynucleotide should be administered in a therapeutically effective amount. A therapeutically effective amount is that amount sufficient to treat the disorder. A therapeutically effective amount can be determined by in vitro experiment followed by in vivo studies. Expression of the inserted polynucleotide can be determined in vitro using any one of the techniques described above. Expression of the inserted polynucleotide can be determined in vivo using any one of several methods known in the art, including immunofluorescence using a fluoresceinated ligand.

The optimal dosage is that which produces maximal improvement with tolerated side effects. It is worth emphasizing that optimal dosage is determined empirically and balances the benefits and adverse side effects.

In another aspect of the present invention, the polynucleotides described above are incorporated into a pharmaceutical composition. A pharmaceutical composition contains a therapeutically effective dose of the construct in a suitable pharmaceutical carrier or diluent. Suitable pharmaceutical carriers and diluents are outlined above. A therapeutically effective dose may be determined by in vitro experiment followed by in vivo studies. The composition may be administered by any one of the methods described above.

The following examples are provided by way of illustration, and not by way of limitation. Unless otherwise indicated, the specific protocols used in the following examples are described in detail in Sambrook et al., Molecular Cloning: A Laboratory Manual, Cold Spring Harbor Laboratory, 1989.

### EXAMPLES

### Example I

Two 30-day-old kittens and two adult felines were deeply anesthetized with 150 mg/kg sodium pentobarbitol (EUTHANYL, Maple Leaf Foods, Inc.) and perfused through the aorta with 1 l of 0.05% diethylpyrocarbonate (DEPC) treated PBS buffer for one minute. Visual cortices, identifiable by observation, were carefully dissected out and quickly frozen either in liquid nitrogen or in a dry ice/isopentane bath for 5 minutes. The dissected tissues were stored at −80° C. until RNA was to be isolated.

### Example II

### Isolation of RNA

a) Total RNA for Northern blot hybridization was isolated using the single steo method of RNA isolation by acid guanidium thiocyanate-ohenol-chloroform extraction method of Chomczynskl and Sacchi. Described in detail in Chomczynski and Sacchi, “Single step method of RNA isolation by acid guanidium thiocyanate-phenol-chloroform extraction,” Anal. Biochem. 162:156-159, 1987, incorporated herein by reference.

b) Poly(A) RNA was isolated directly from the tissue of interest using a FAST TRACK mRNA isolation kit (Invitrogen Corporation) or QUICK mRNA PREP (Pharmacia).

### Example III

### Construction of the cDNA Library

An adult visual cortex plasmid library in pcDNAII (Invitrogen Corporation) was constructed by a modified Gubler-Hoffman procedure, described in detail in Gubler and Hoffman, “A simple and very efficient method for generating cDNA libraries,” Gene 25:263-269, 1983, incorporated herein by reference. Briefly, the procedure utilizes the synthesis of first strand cDNA using AMV reverse transcriptase (Life Sciences), cleavage of mRNA hybrid using E. coli RNase H (BRL), and synthesis of second strand cDNA using E. coli DNA polymerase I. Any nicks in the double-stranded cDNA were repaired using E. coli DNA ligase (BRL) The double-stranded cDNA was blunt ended using T4 polymerase (BRL) BstXI non-palindromic linkers (Invitrogen Corporation) were ligated to the double-stranded, blunt-ended cDNA. Following the removal of small length cDNAs and excess linkers using size select 400 columns (Stratagene) the cDNA was ligated to the BstXI cut pcDNA II vector and transformed in DH5α MAX Efficiency competent cells (BRL).

### Example IV

### Preparation of a Kitten

### Visual Cortex Specific Probe

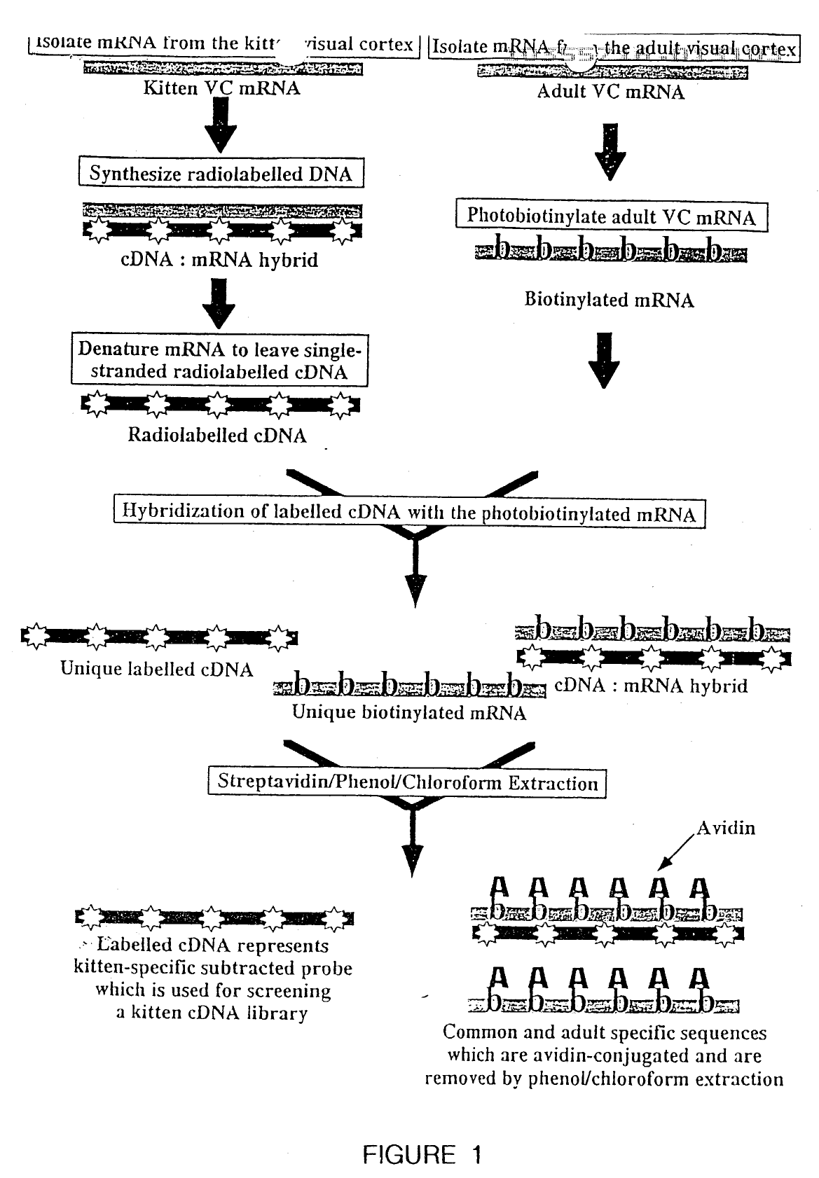

In order to obtain the cDNA clones representing the mRNA enriched in the 30-day-old kitten visual cortex relative to the adult visual cortex, a 30-day-old kitten visual cortex cDNA probe was prepared. The strategy used in the synthesis of the subtractive probe is outlined in FIG. 1, and described in detail in Sieve and St. John, “A simple subtractive hybridization technique employing photoactivatable biotin and phenol extraction,” Nucl. Acids Res. 16:10937, 1988, incorporated herein by reference.

a) Biotinylation of Adult mRNA

Twenty μg of the adult visual cortex poly(A) RNA in 30 μl sterile diethylpyrocarbonate treated water (DEPC water) was combined with an equal volume of photobiotin acetate (1 mg/ml) in a 1.5 ml screw cap tube in the darkroom. The tube with its cap closed was placed in ice slurry exactly 10 cm below a 300W reflector lamp for 30 minutes. The mixture was then diluted to 200 μl with 0.1 M Tris, pH 9, followed by extractions with equal volumes of water saturated 2-butanol until the butanol layer appeared clear. The biotinylated adult visual cortex mRNA was then ethanol precipitated, washed with 80% ethanol and dissolved in 30 μl of sterile RNase free water. The photobiotinylation procedure was repeated to achieve a more efficient biotinylation.

b) Synthesis of Labeled cDNA:

A [³²P]-labeled cDNA probe was synthesized from 1 μg of poly(A) RNA derived from the 30-day-old kitten visual cortex using oligo (dt) as a primer and 250 μCi of [a-³²P] dCTP (3000 Ci/mmol; 1 Ci=37 GBq). This resulted in the antisense cDNA population for the kitten visual cortex with a total incorporation of 10⁸ cpm. The mRNA template was removed by hydrolysis with 0.5 M NaOH at 55° C. for 15 minutes. The labeled cDNA (approximately 500 ng) was precipitated with ethanol and used immediately in the subtraction step with the biotinylated adult visual cortex mRNA.

c) Subtractive Hybridization:

In a screw cap vial, the 30 μl of biotinylated adult visual cortex mRNA (10 μg) was mixed with the [³²P]-labeled 30-day-old kitten visual cortex antisense cDNA (0.5 μg) and ethanol precipitated. The final pellet was resuspended in 10 μl of sterile water and 10 μl of 2×hybridization buffer (0.65 M NaCl, 0.04 M Na₂PO₄, pH 6.8, 1 mM EDTA and 0.05% SDS) was added. This mixture was boiled for 1 minute and then incubated at 65° C. for 48 hr. Under these conditions, Cot values greater than 1000 were obtained. To the hybridization mixture 30 μl of 10 mM Hepes/EDTA buffer was added followed by the addition of 10 μl of 1 mg/ml of streptavedin. This mixture was mixed and incubated at room temperature for 10 minutes and then extracted with 60 μl of phenol/chloroform. The organic phase was back extracted with 50 μl of Hepes/EDTA buffer and aqueous layers were combined. Another 10 μl of streptavedin was added to the aqueous phase and the mixture was incubated at room temperature for 5 minutes. This mixture was again extracted with an equal volume of phenol/chloroform and precipitated with ethanol. The final pellet was resuspended in sterile RNase free water and hybridized with a second aliquot of biotinylated adult visual cortex mRNA (10 μg) as described above. The labeled single stranded cDNA remaining after two rounds of subtraction represented 3%-10% of the starting material.

### Example V

### Screening of cDNA Library

Approximately 12,000 independent cDNA clones derived from a 30-day-old visual cortex cDNA library were plated on ampicillin plates at a density of 1,000 clones/plate. The colonies were allowed to grow to about 0.5 mm in diameter and a sterile nylon membrane was carefully placed on each plate with proper identifiable orientation. The plates were incubated for 2 additional hours. The filters were prehybridized in 2×SCC, 1% SDS, 0.5% nonfat dry milk, pH 7.0 for 1 hour at 65° C. The filters were then removed and hybridized with the subtractive probe (200,000 cpm/ml) in a solution containing 6×SSC, 1% SDS, 0.5% nonfat dry milk, pH 7.0 at 65° C. for 16 hours. The post hybridization washes were done at 50° C. in 0.1×SSC, 1% SDS, pH 7.0. The filters were then exposed to Kodak XAR 5 film for 6 hours. The positive clones were picked up and stored in 96 well microtiter plates containing 200 μl of 20% glycerol in Luria broth medium.

### Example VI

### Synthesis of RNA Probes

The plasmid DNA was isolated by the alkaline lysis minipreparation method. The plasmid DNA was linearized by digestion with either XhoI or HindIII. Depending upon the orientation of the cDNA inserts, the corresponding antisense riboprobes were prepared using either the SP6 RNA polymerase or the T7 RNA polymerase with [³²P]CTP following the instructions in the PROMEGA kit (Promega Corp.).

### Example VII

### Northern Blot Hybridization

10 μg of various total RNAs (determined by A₂₆₀ spectrophotometric measurements) were electrophoresed on a 1.1% agarose gel containing 0.66 M formaldehyde at 4V/cm for 4 to 4.5 hours. RNA from the gel was transferred to a nylon based membrane (Gene Screen, NEN) using the procedure described by Thomas, “Hybridization of denatured RNA and small DNA fragments transferred to nitrocellulose,” Proc. Natl. Acad. Sci. USA 77:5201-5205, 1980, incorporated herein by reference. After transfer of the RNA to nylon membranes, the membrane was exposed to a UV transilluminator for 1 min. Khandjian, “UV crosslinking of RNA to nylon membranes enhances hybridization signals,” Molec. Biol. Rep. 11:107-115, 1986, incorporated herein by reference. The filters were baked at 80° C. for 1-2 hr. Prehybridization was carried out for 30 min. in S0% deionized formamide, 0.25M sodium phosphate, pH 7.2, 0.25M NaCl, 1 mM EDTA, 7% SDS and 5% polyethanol glycol (MW 8000). Hybridization was performed in the same buffer with the inclusion of 1×10⁶ cpm/ml. of the individual riboprobes. Four 20 min. post hybridization washes were carried out in 0.04M sodium phosphate, pH 7.2, 1% SDS, 1 mM EDTA at 65° C. The membranes were then rinsed three times in 2×SSC for 5 min. at room temperature followed by 15 min. treatment with 2×SSC containing 1 μg/ml of RNase A. The filters were rinsed for 30 min. at 500° C. in 0.1×SSC, 0.1% SDS and then exposed to Kodak XARS films for 1-3 days, depending on the desired Intensity, in the presence of an intensifying screen.

### Example VIII

### DNA Sequence Analysis

The plasmid DNA was isolated by the alkaline lysis minipreparation method. Approximately 5 μg 35 supercoiled plasmids were further purified by passing them through Plasmid-Quick columns (Stratagene, Corp.) and then subjected to the dideoxy sequencing. Sanger et al., “DNA sequencing with chain-terminating inhibitors,” Proc. Natl. Acad. Sci. USA 74:5463-5467, 1977, incorporated herein by reference. Each purified template was sequenced at least twice either manually using a SEQUENASE Version 2.0 kit (USB) or using a model number 373A ABI automated DNA sequencer. The sequencing reactions for the automated ABI DNA sequencer were performed using a PERKIN-ELMER thermal cycler with the annealing of dye-labeled universal M13 forward or reverse primers. In some instances the sequencing reactions were also carried out using the dye terminators.

### Example IX

### Identification of Gene Sequences

The FASTA, Pearson and Lipman, “Improved tools for biological sequence analysis,” Proc. Natl. Acad. Sci. USA 85:2444-2448, 1988, incorporated herein by reference, and BLAST, Altshul et al., “Basic local alignment search tool,” J. Mol. Biol. 215:403-413, 1990, incorporated herein by reference, sequence programs were used for sequence data compilation and to search for sequence identity, in both the nucleic acid and amino acid coding domains. Some of the searches were carried out at the National Centre Of Biotechnology Information (NCBI) using the BLAST network service. Sequences were aligned and formatted using the eyeball sequence editor program, ESEE. Cabot and Beckenbach, “Simultaneous editing of multiple nucleic acids and protein sequences with ESEE,” Comput. Applic. Biosci. 5:233-234, 1989, incorporated herein by reference.

Isolation of cDNA Clones:

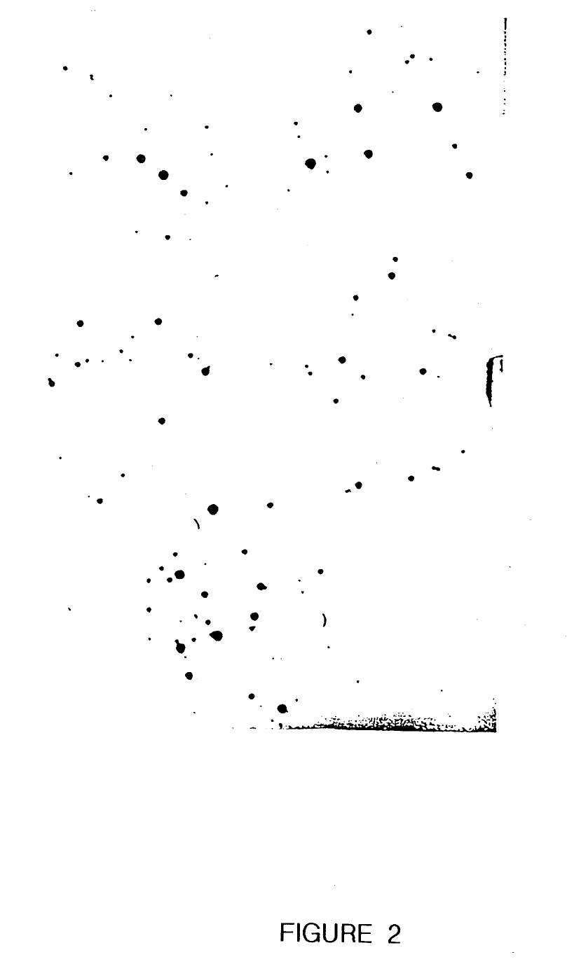

Approximately 12,000 independent cDNA clones derived from a cDNA library for the 30 day old kitten visual cortex were screened with a kitten visual cortex specific cDNA probe. This probe was prepared in such a way that the sequences also expressed in the adult visual cortex were removed by subtractive hybridization. 200 of the total screened clones hybridized to the subtracted probe (FIG. 2). As the signal intensities are likely to represent the abundance level of the corresponding mRNA, the clones with strong signal as well as those with weak signal were isolated and individually grown for miniplasmid preparations. The plasmid DNA was isolated from each clone and digested with XhoI and HindIII restriction endonuclease to release the cDNA inserts. The digested DNA was electrophoresed on 1% agarose gels and bidirectionally blotted to nylon membranes using the procedure of Smith and Summers. Smith and Summers, “The bidirectional transfer of DNA and RNA to nitrocellulose or DBM paper,” Anal. Biochem. 109:123-129, 1980, incorporated herein by reference. These blots were then probed with gel-purified twenty cDNA inserts (randomly chosen) radiolabelled by the oligo-labeling procedure. Feinberg et al., “A technique for radiolabeling DNA restriction endonuclease fragments to high specific activity,” Anal. Biochem. 132:6-13, 1983, incorporated herein by reference. This was done to determine if any of the isolated cDNA clones were in multiple copies. One cDNA clone pKVC18 hybridized to twenty different cDNA clones and was therefore present in 10% of the purified cDNA clones. Seventy five percent of these clones were found to be represented in single copy and the remaining 15% were in two to four copies (data not shown).

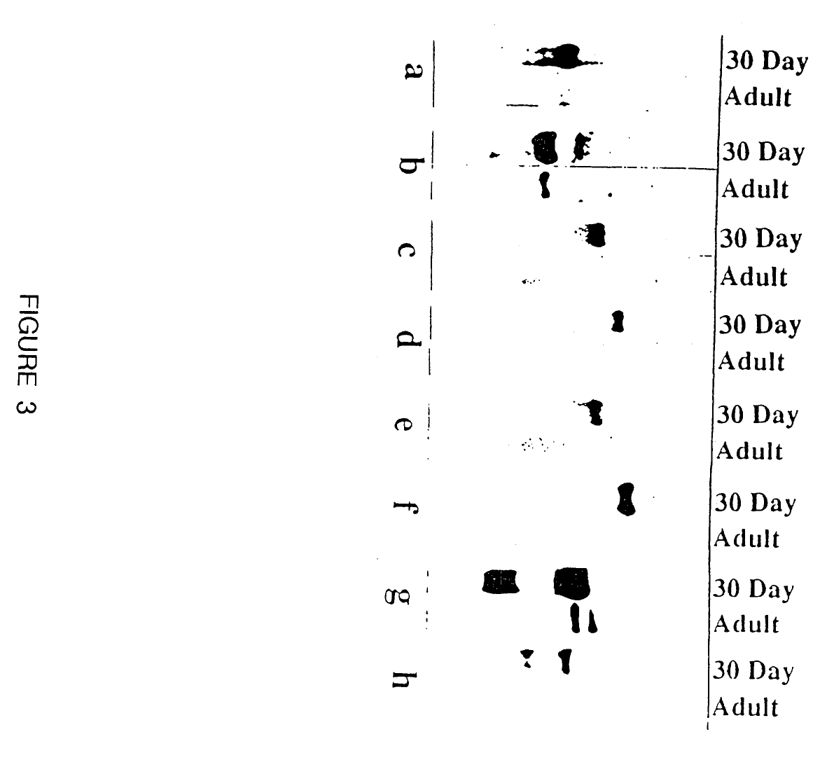

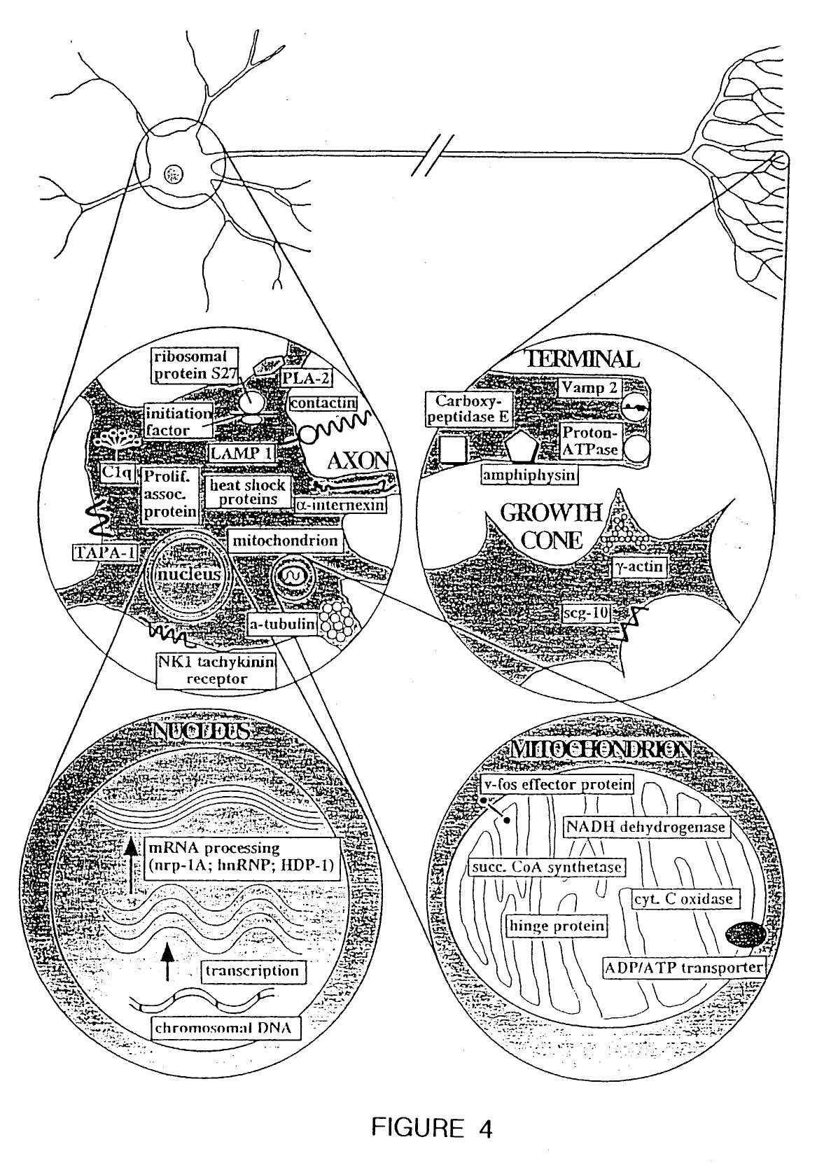

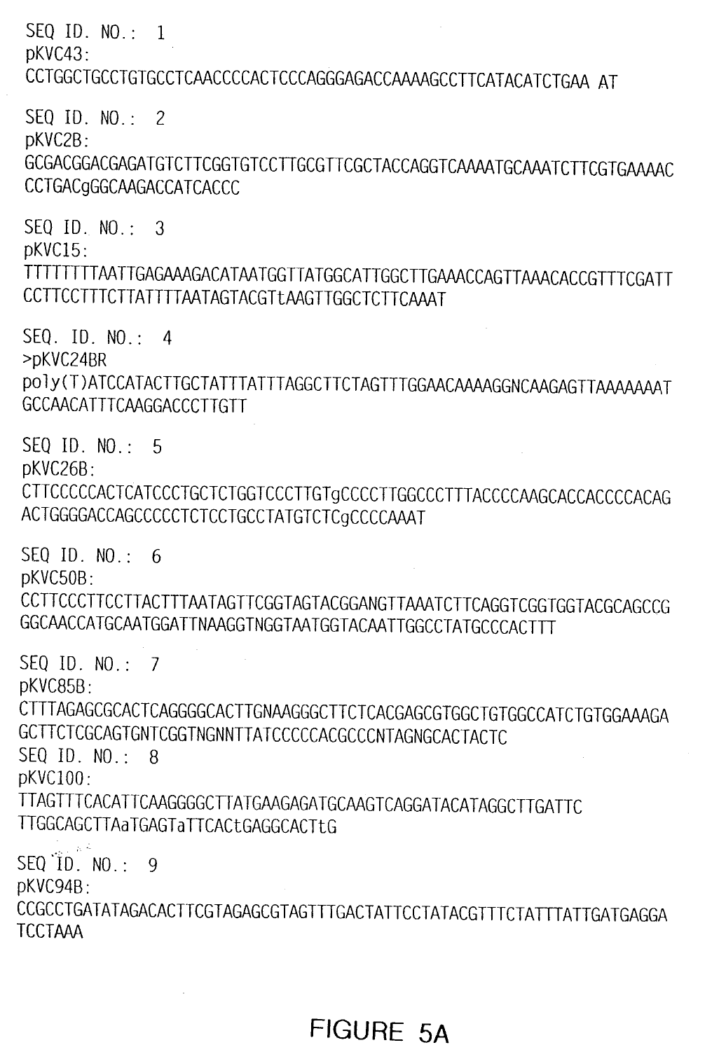

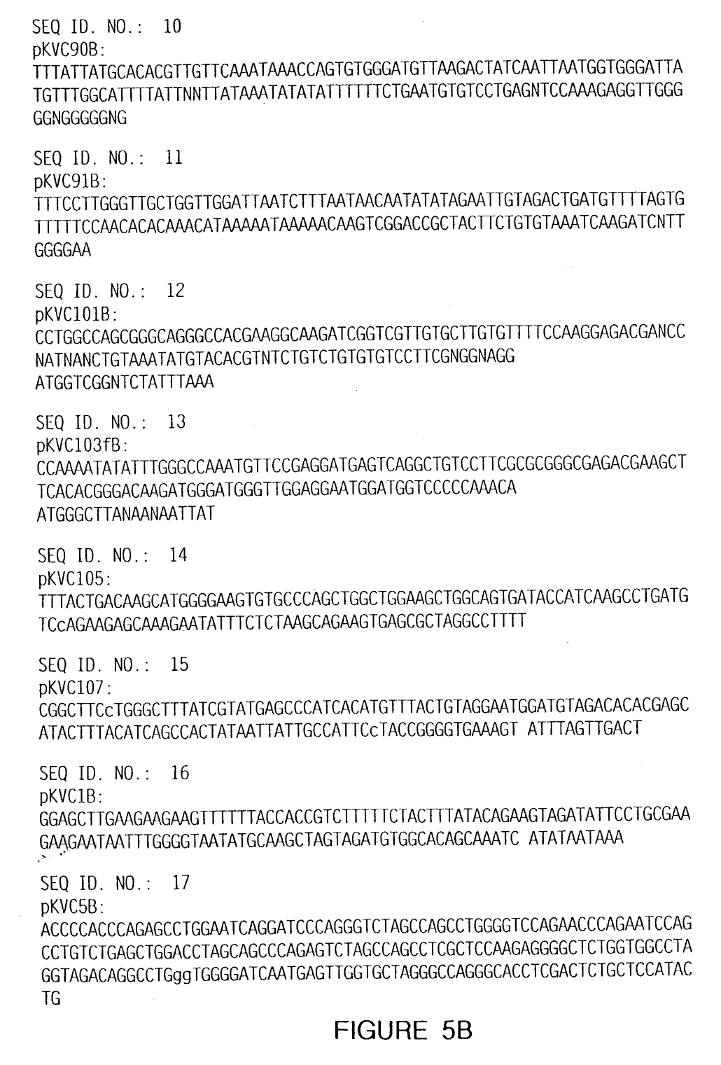

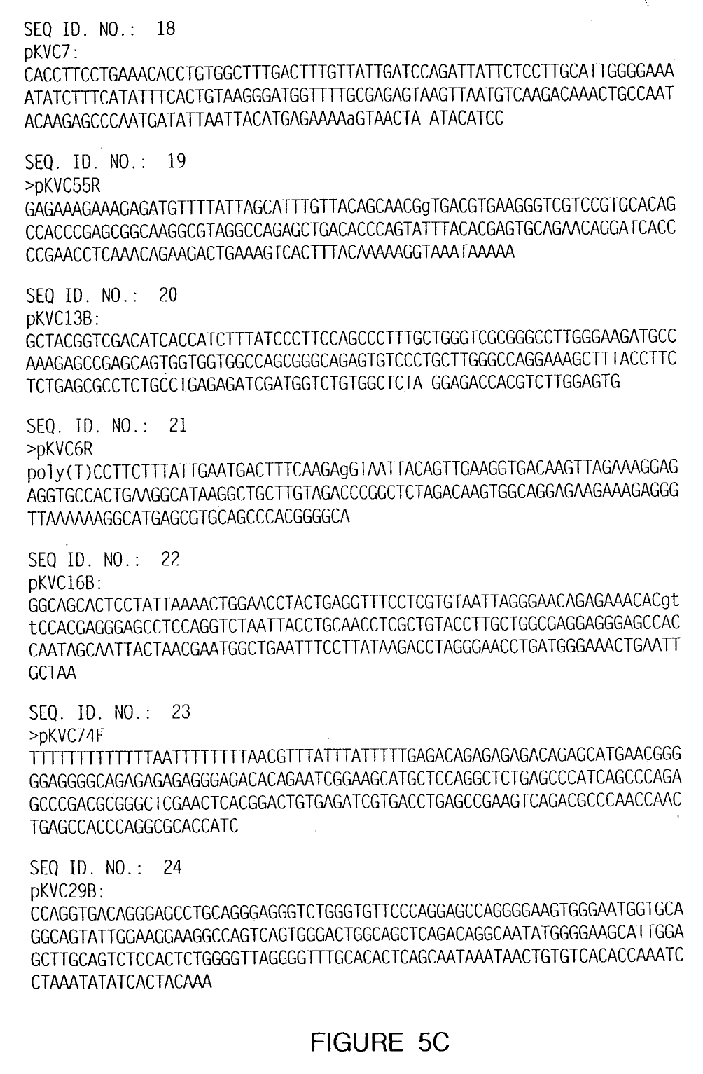

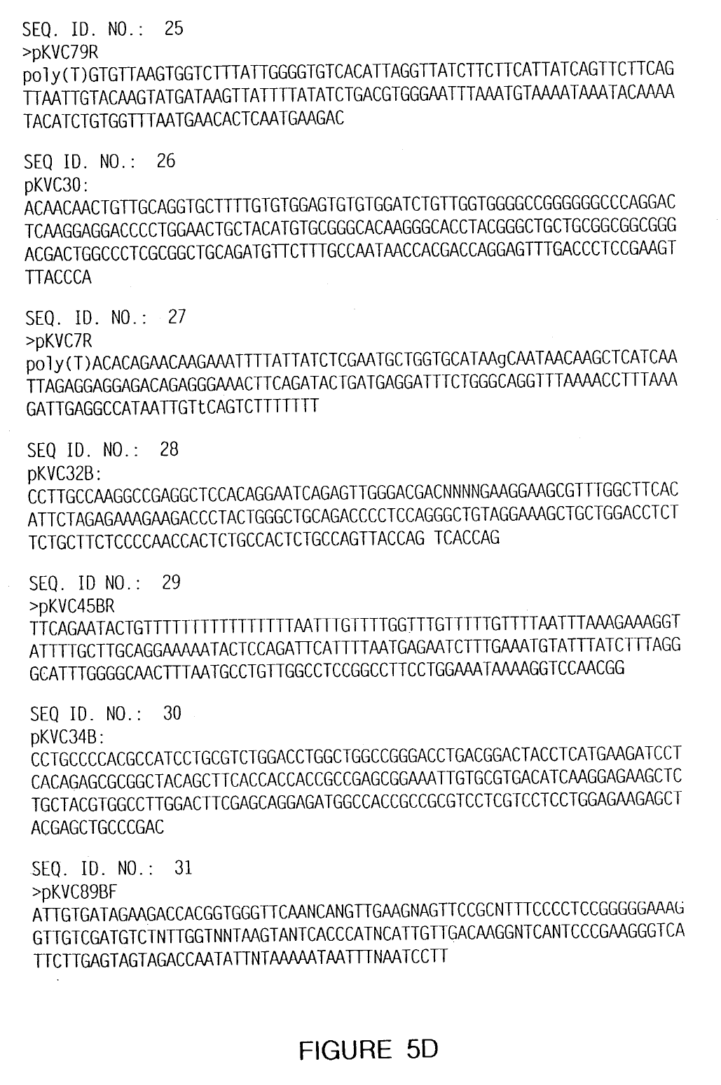

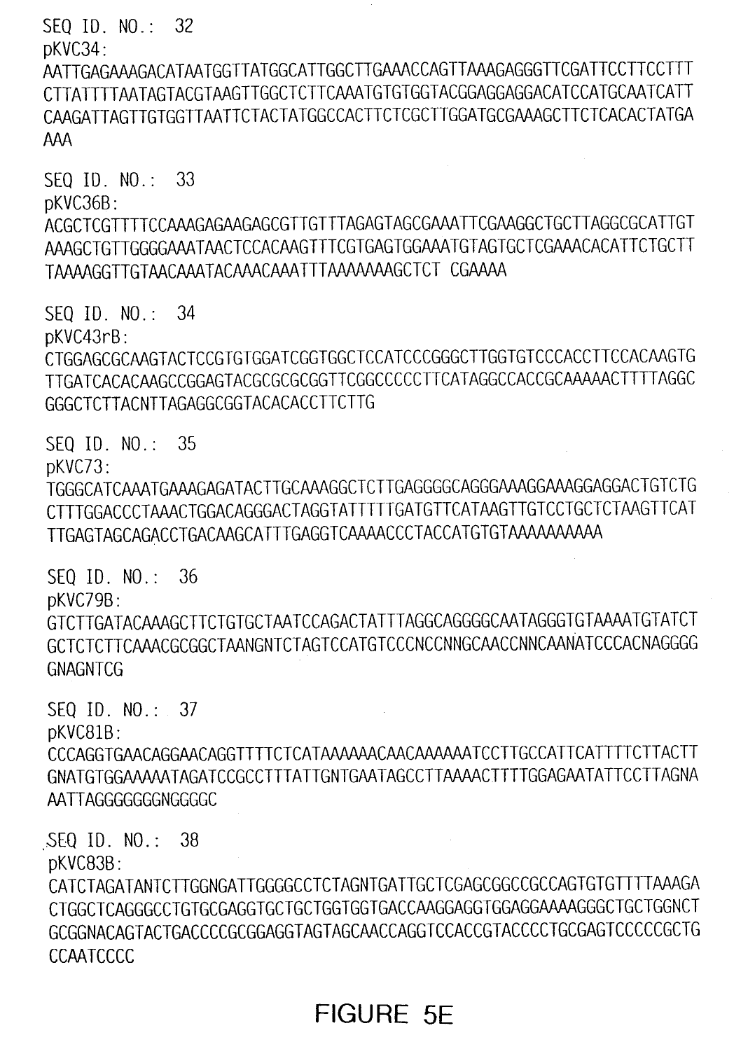

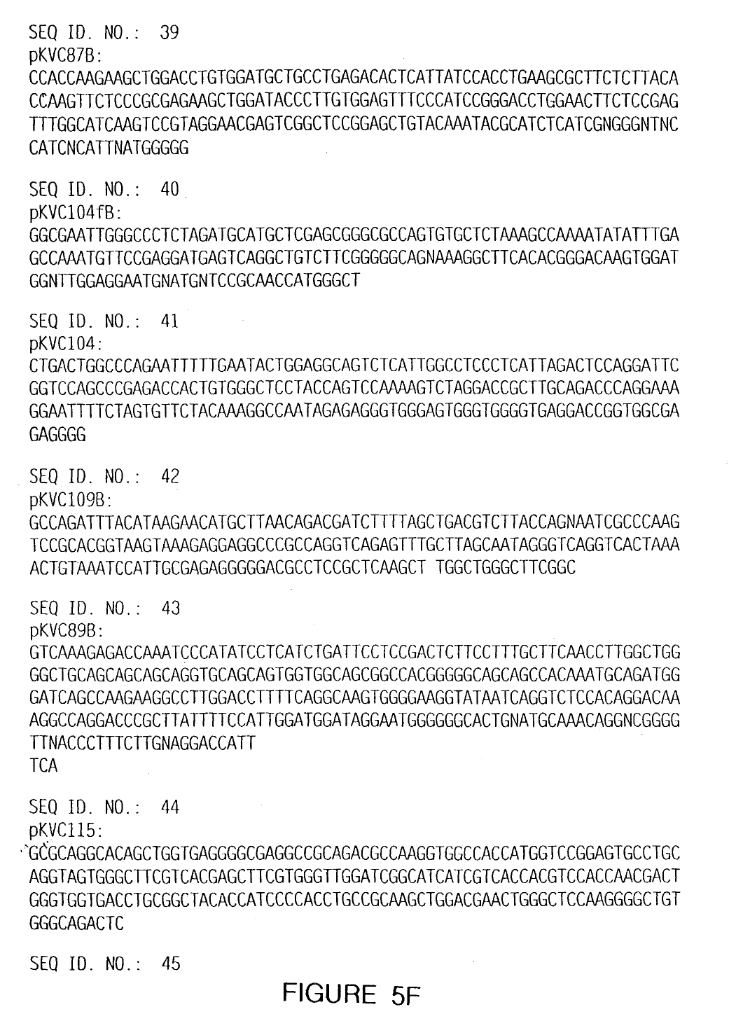

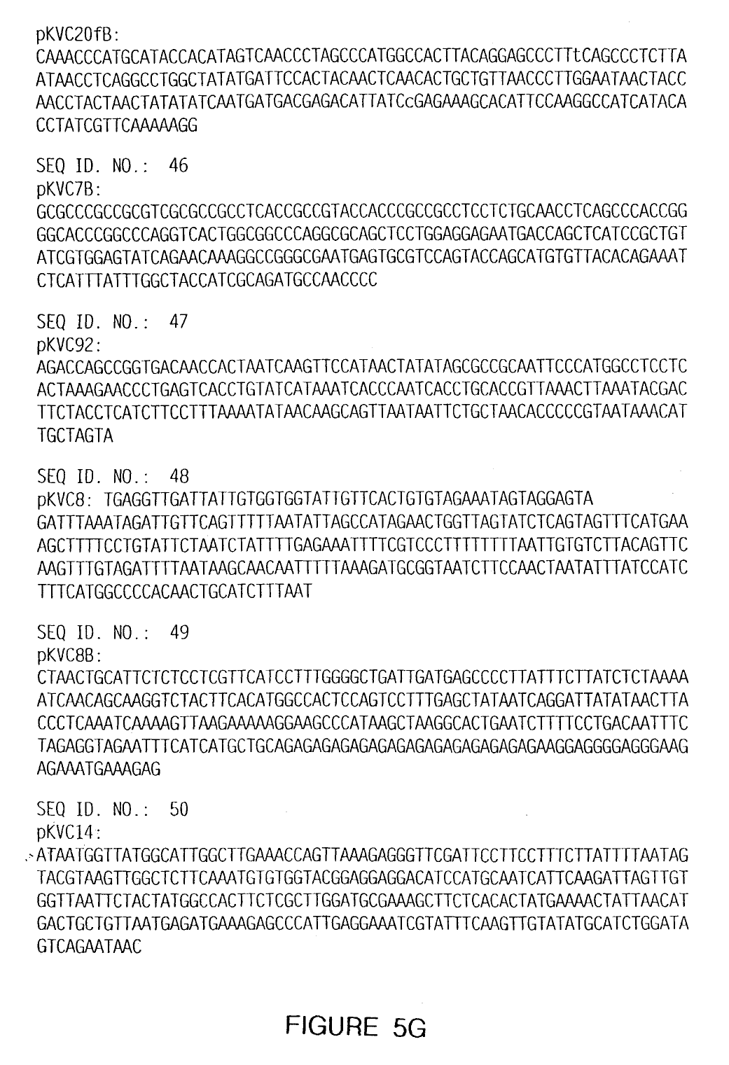

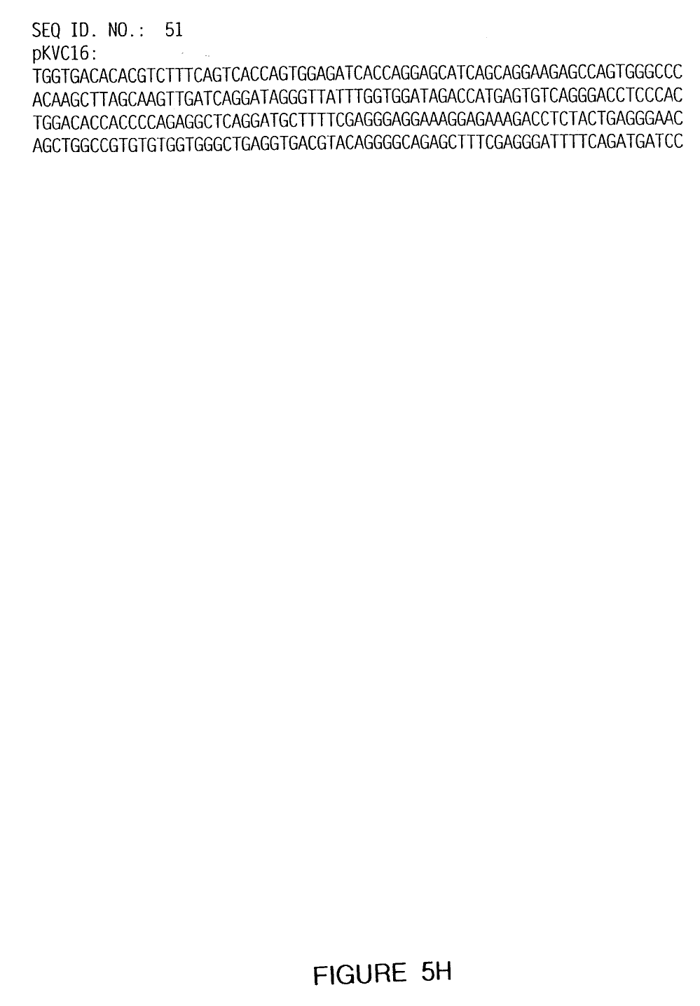

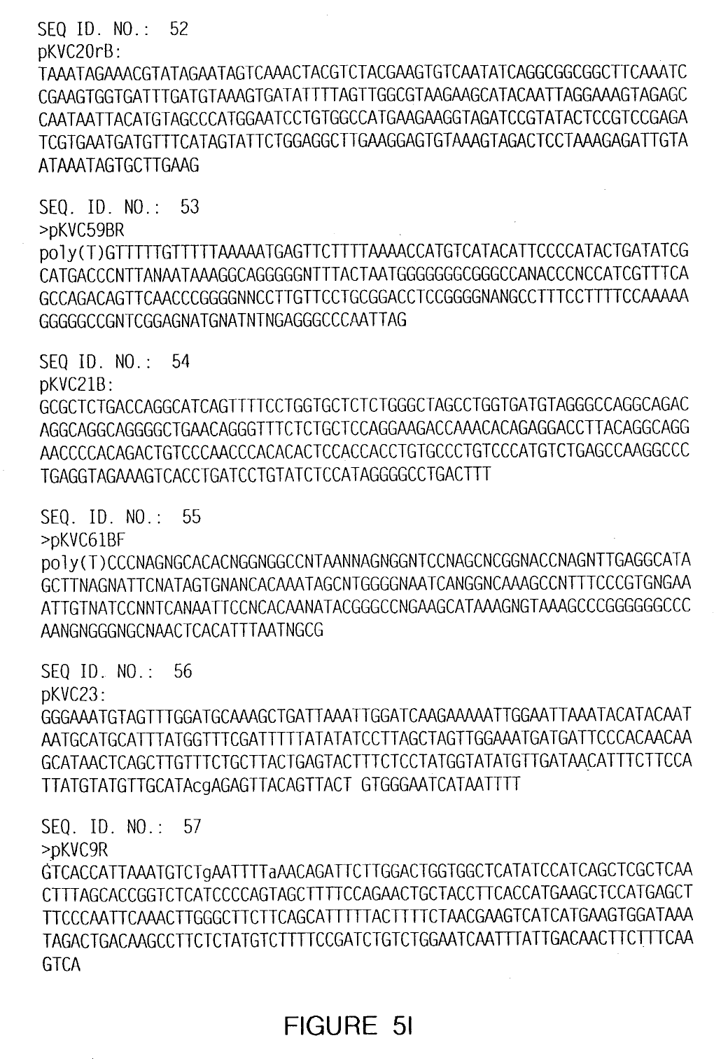

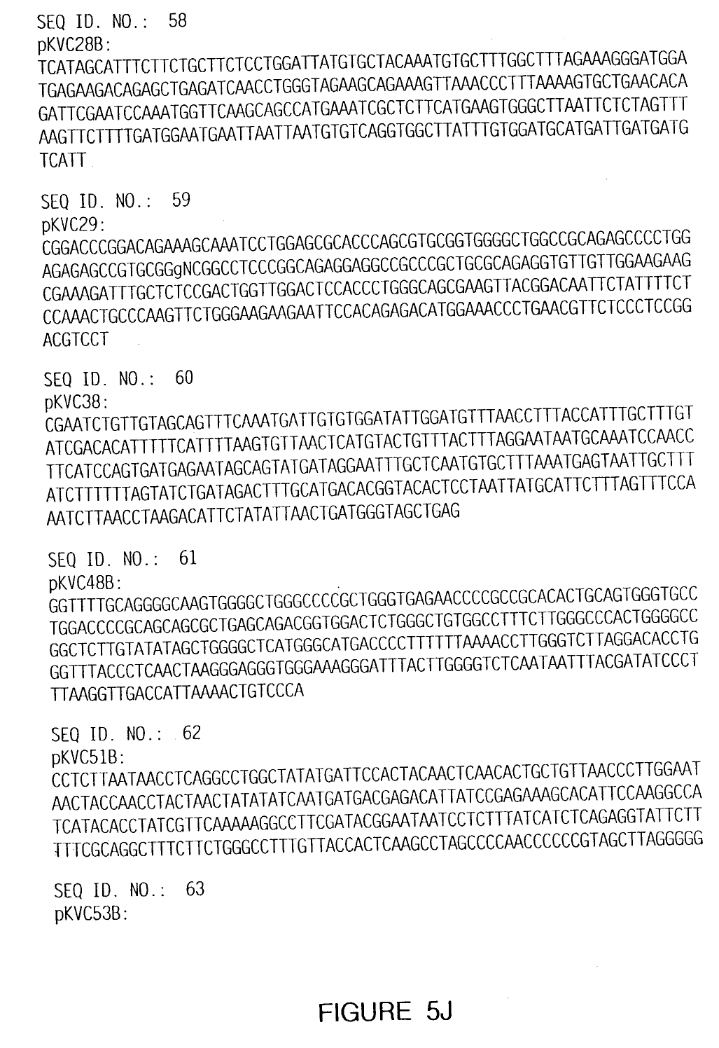

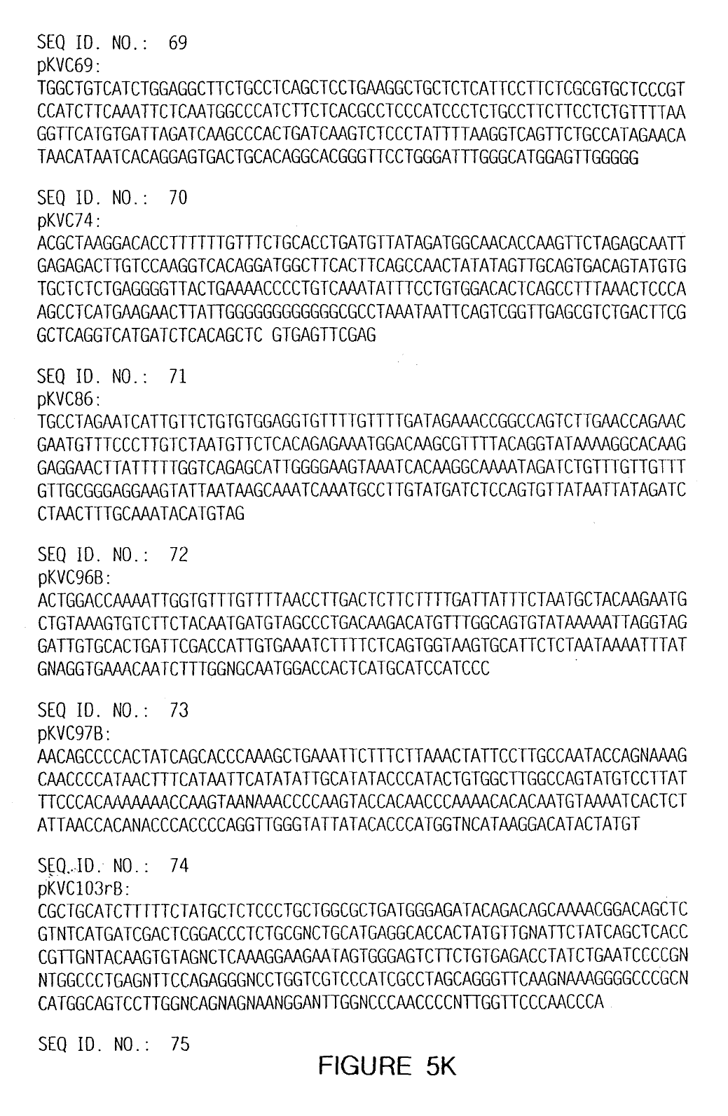

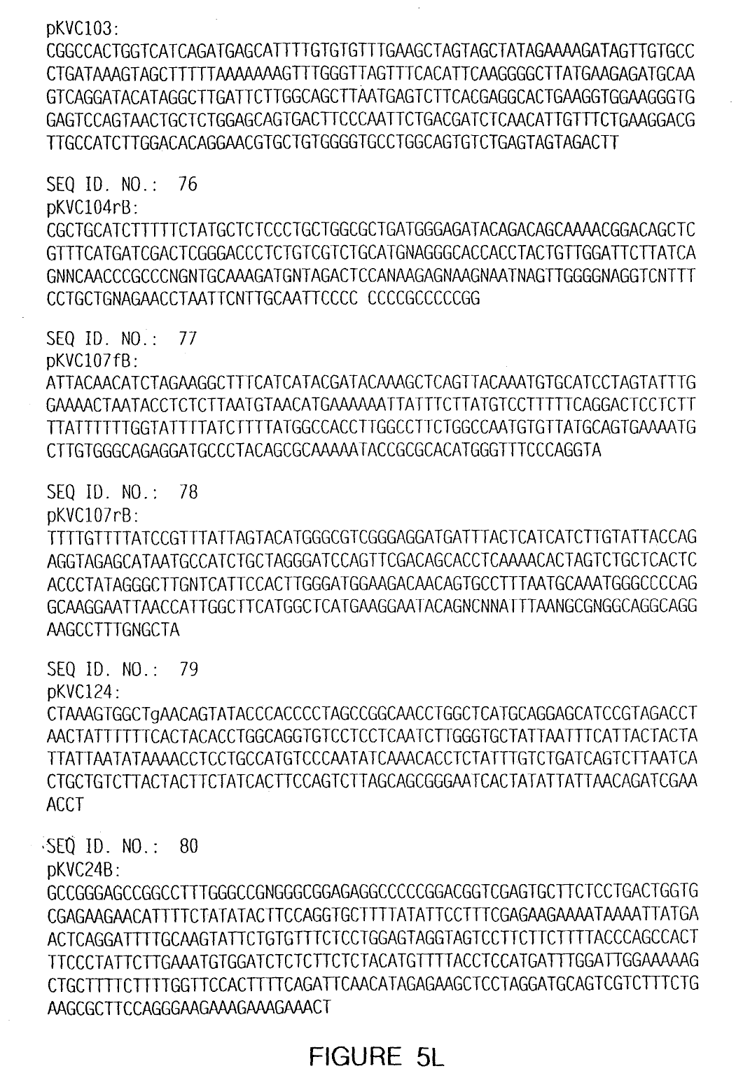

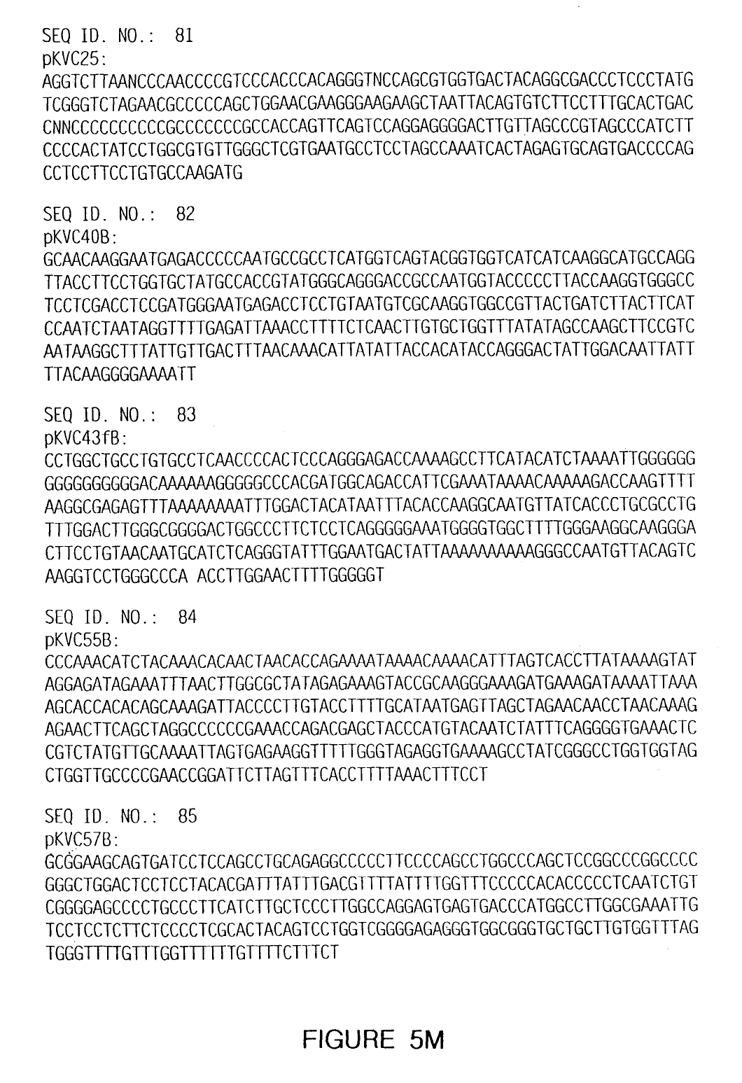

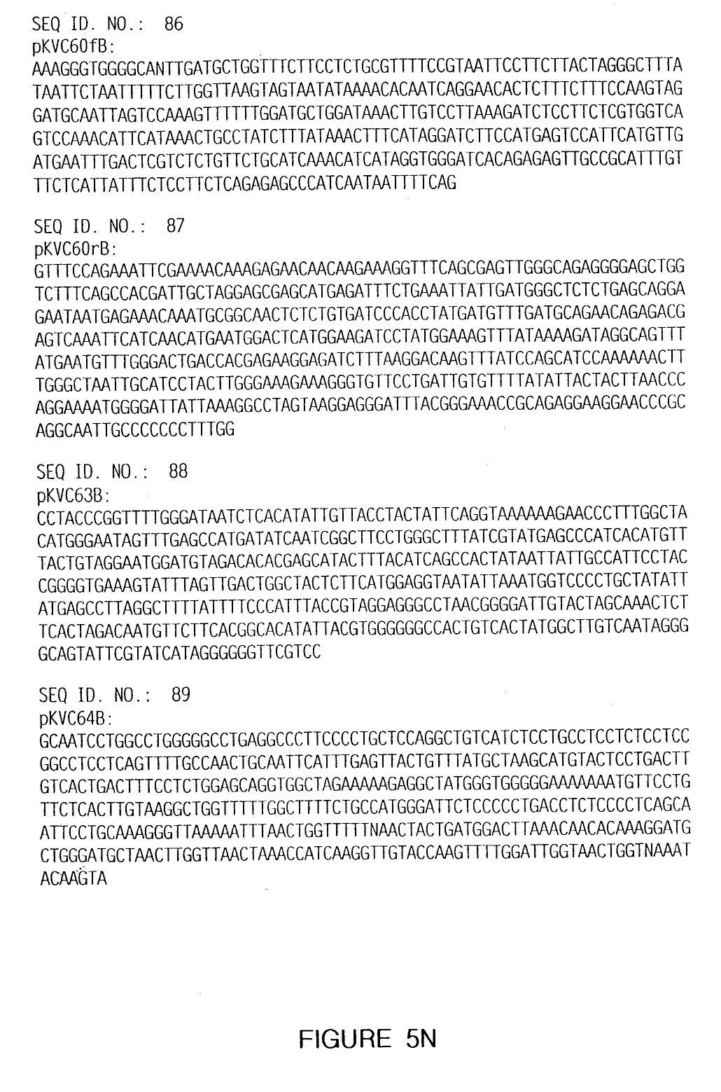

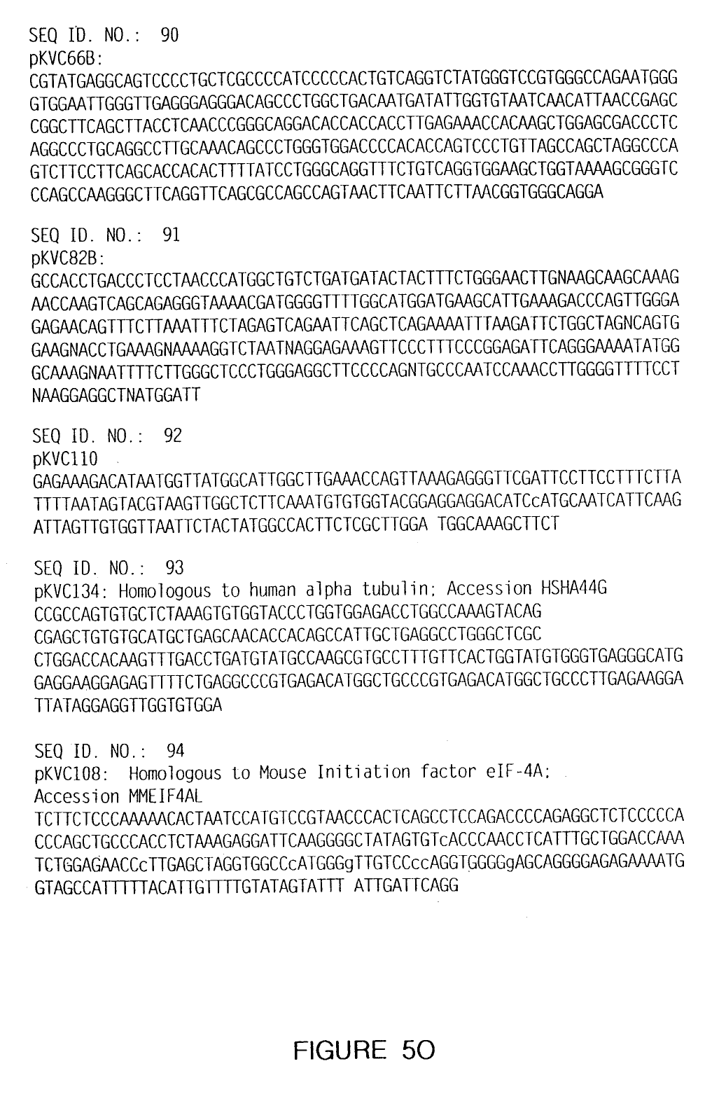

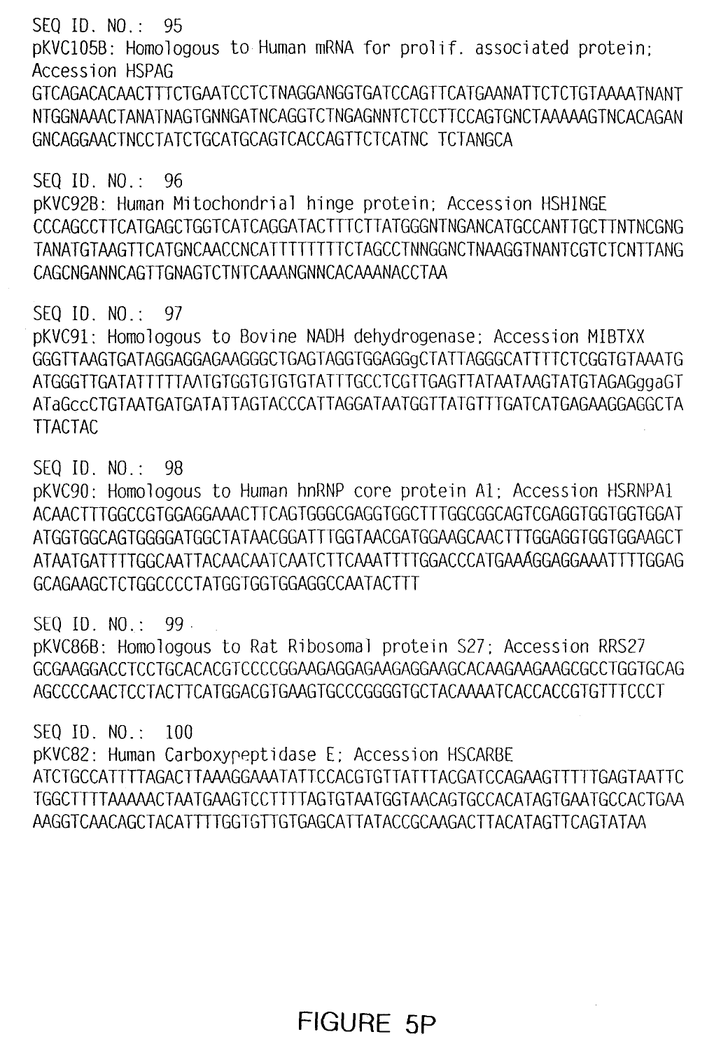

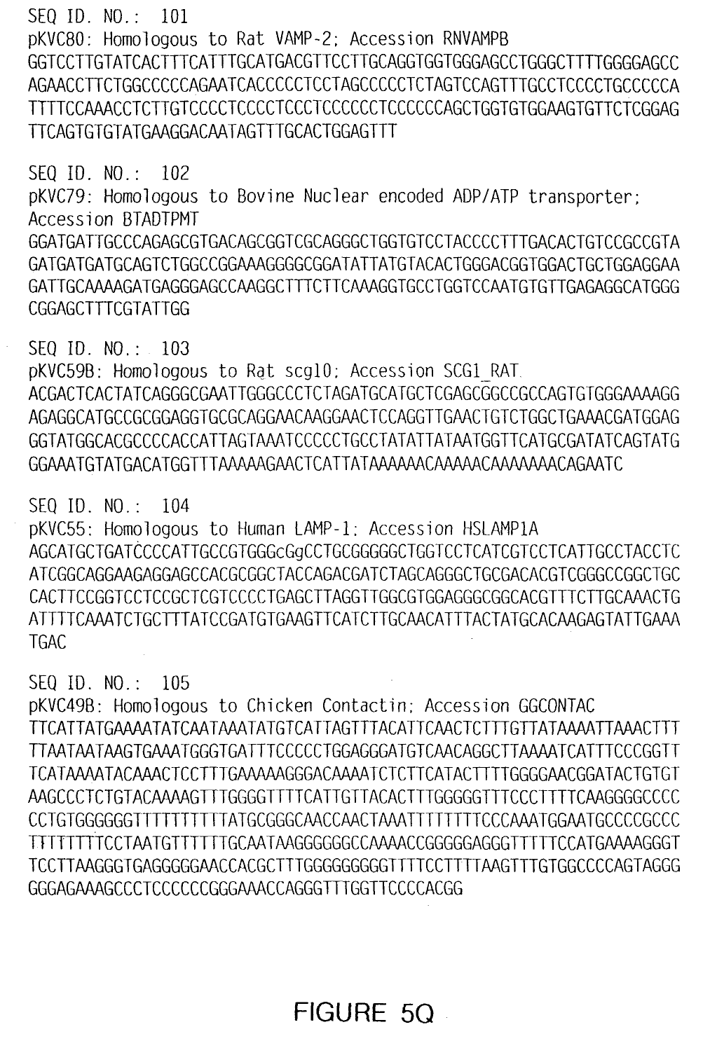

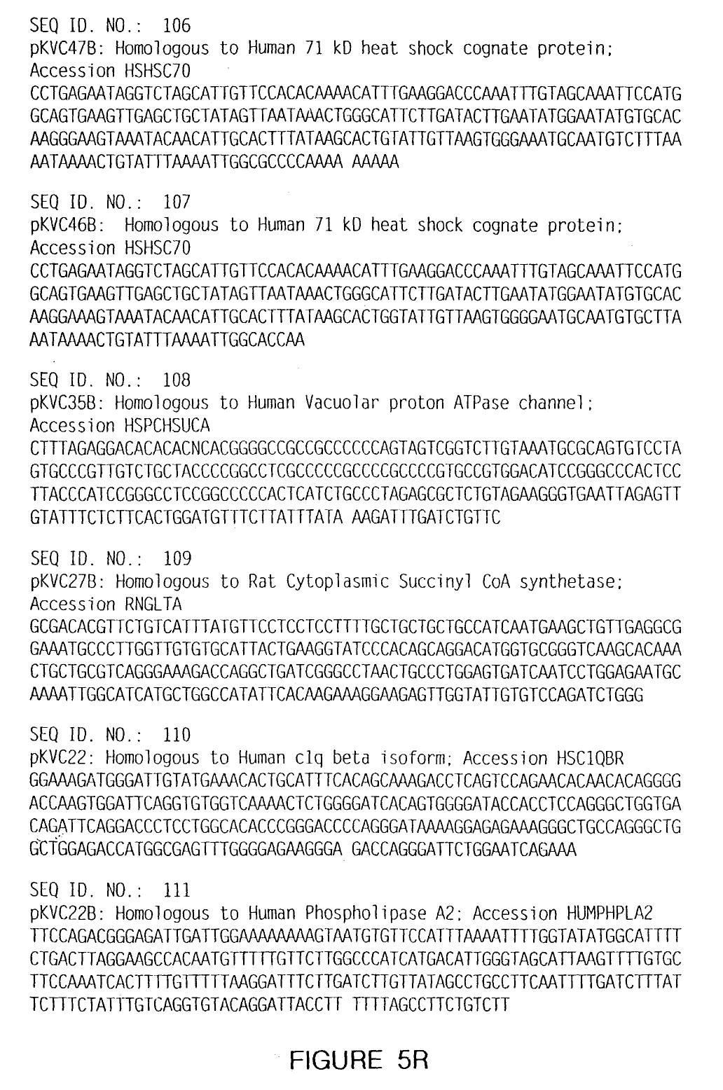

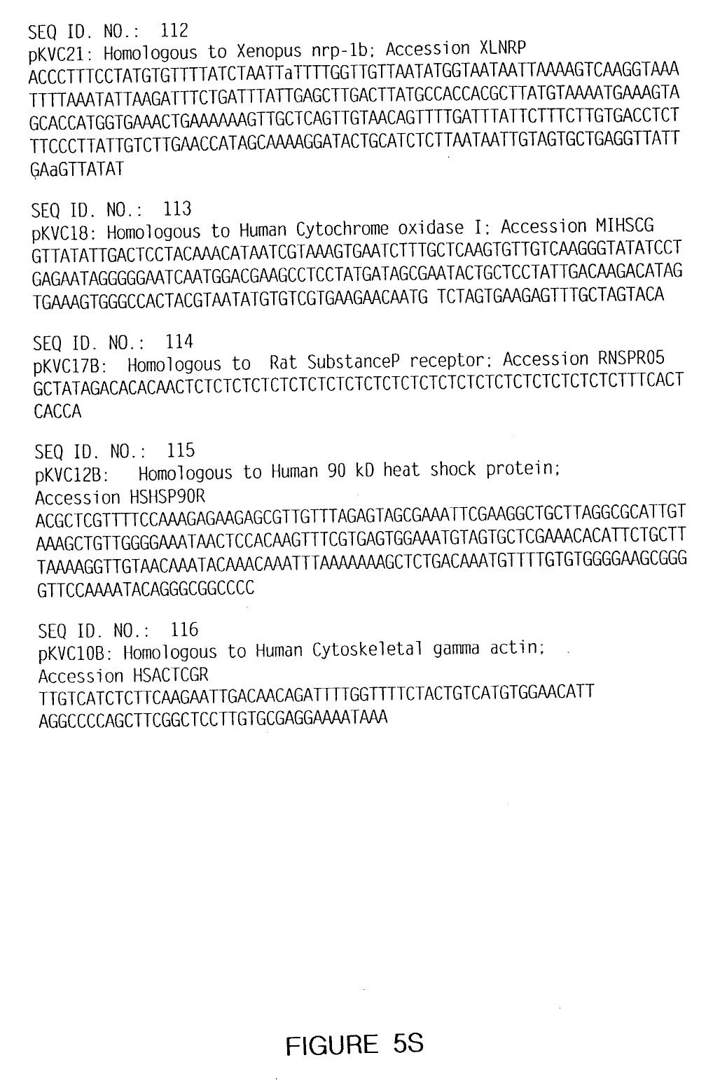

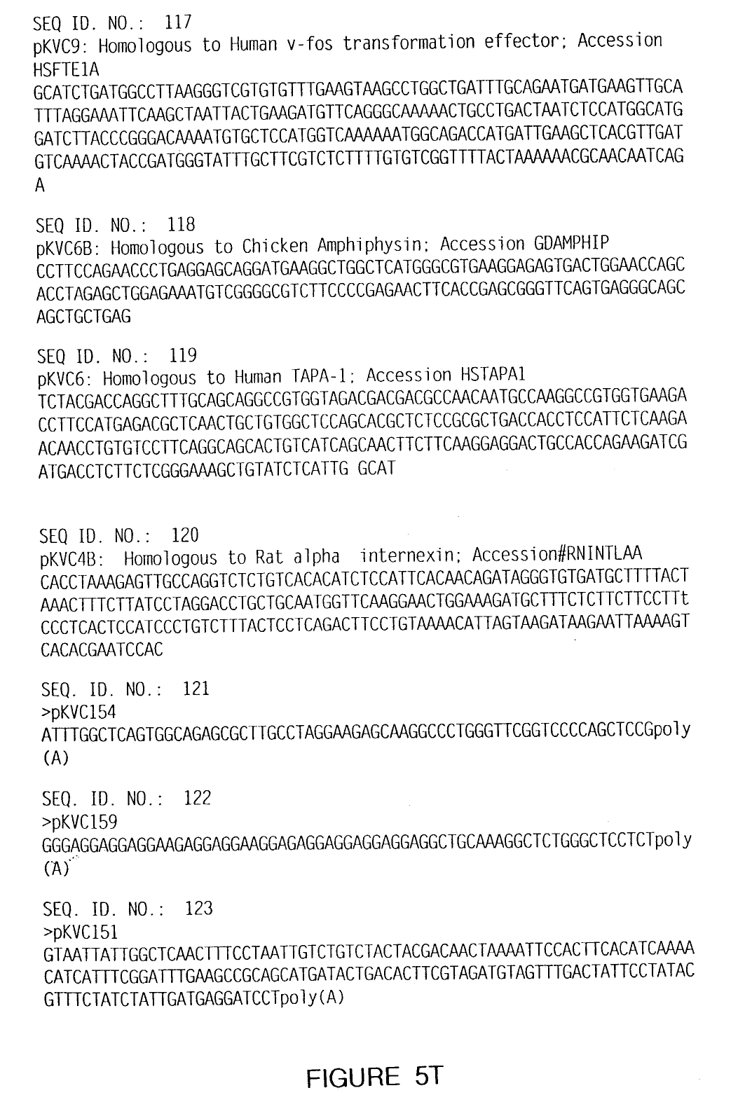

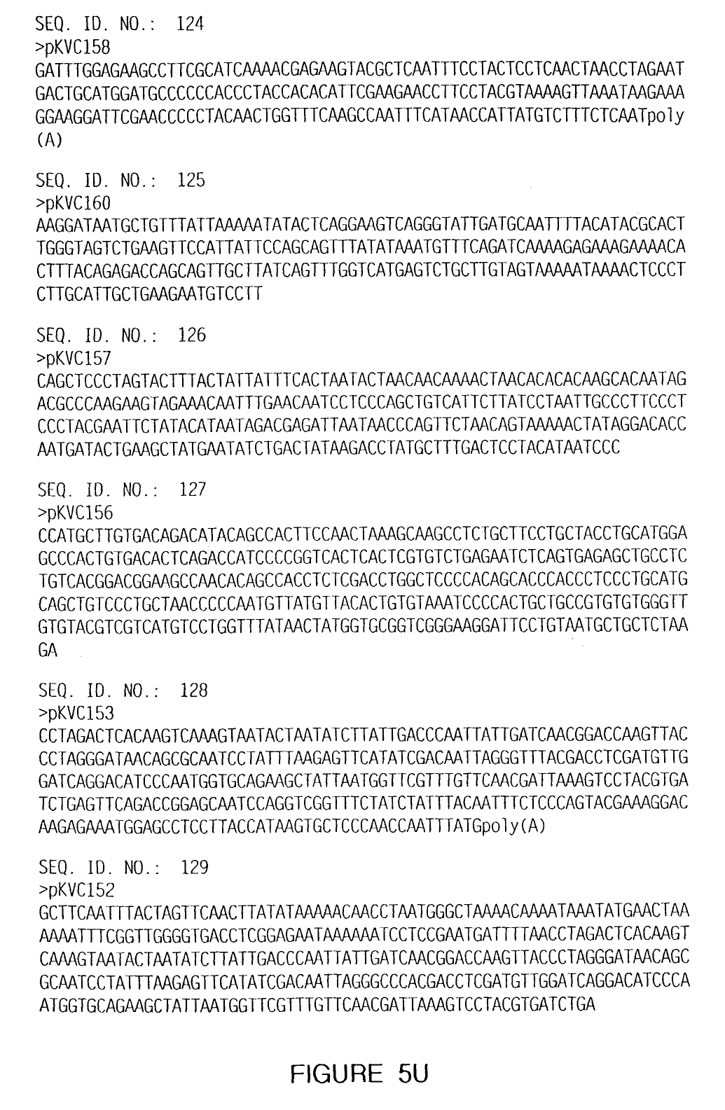

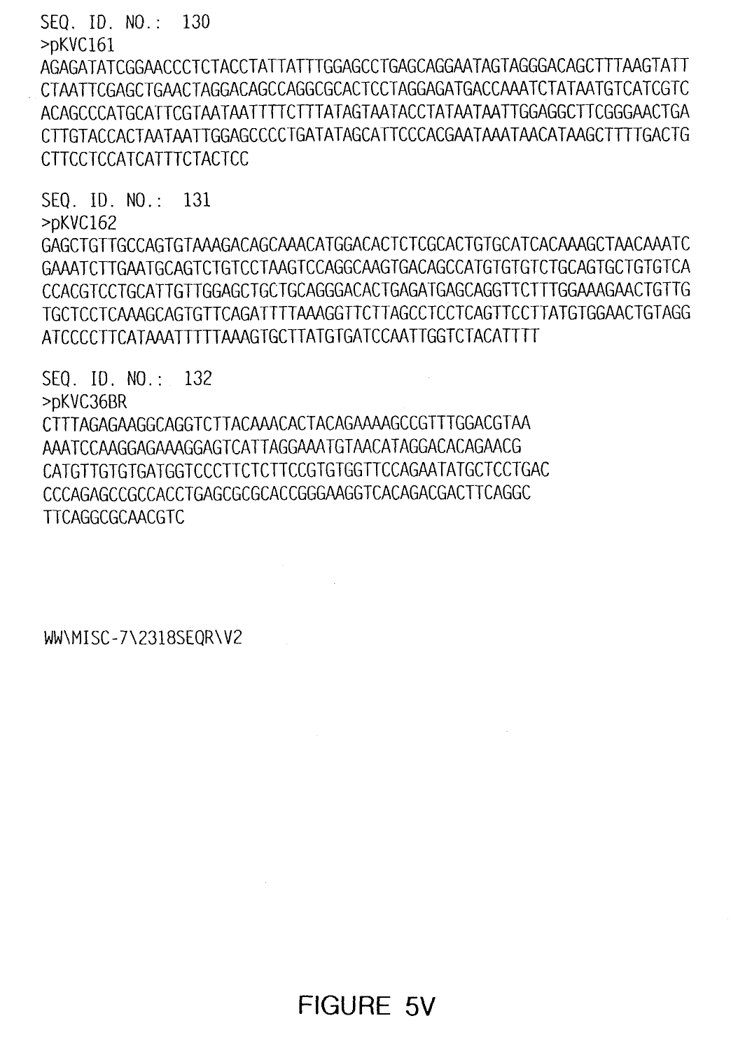

In order to verify whether the identified clones were truly representative of the mRNA abundance in the visual cortex of kittens and felines, we tested 50 of these clones using Northern blot hybridization. Total RNA was prepared from the visual cortex derived from a different group of 30-day-old kittens and adult felines. Fifteen micrograms of each RNA was electrophoresed through 1.1% agarose gels containing 0.66 M formaldehyde and blotted to nylon membranes using the procedure described by Thomas. Thomas, “Hybridization of denatured RNA and small DNA fragments transferred to nitrocellulose,” Proc. Natl. Acad. Sci. USA 77:5201-5205, 1980, incorporated herein by reference. The blots were hybridized with the riboprobes synthesized from the individual cDNA clones. Among the 50 tested clones only one clone was found to be expressed in similar levels in kitten and adult cat visual cortex. As shown in FIG. 3 for eight of these clones, the hybridization signals were much more intense in the lanes containing the 30-day-old kitten visual cortex mRNA relative to those in the lanes containing the adult visual cortex mRNA. Probing a similar blot with a ubiquitin probe (obtained from a cat visual cDNA library) confirmed the uniform loading, transfer and integrity of both the kitten as well as adult visual cortex RNAs (data not shown). The band intensity for each lane in the resulting autoradiogram was scanned using the NIH Image program (version 1.46). Comparison of the band intensities for each probe in the lane containing kitten visual cortex mRNA with that of the adult visual cortex mRNA indicated that the signals were either unique to or at least 4- to 25-fold higher in the kitten visual cortex mRNA. This indicates that these clones detected RNAs that were either entirely specific to the 30-day-old kitten visual cortex at the sensitivity of Northern blot analysis or were at least highly enriched in the kitten visual cortex in comparison to the adult visual cortex.

We were interested in identifying the encoded product for these cDNA clones. Therefore we determined the sequence of approximately 400 nucleotides from both ends of 132 of these cDNAs. The open reading frame was identified at one end for 90 of these clones and translated to amino acids using the eyeball sequence editor (ESEE) program. Cabot and Beckenbach, “Simultaneous editing of multiple nucleic acids and protein sequences with ESEE,” Comput. Applic. Biosci. 5:233-234, 1989, incorporated herein by reference. The amino acid sequence was used to search the EMBL release 28 DNA database using the FASTA program. Pearson and Lipman, “Improved tools for biological sequence analysis,” Proc. Natl. Acad. Sci. USA 85:2444-2448, 1988, incorporated herein by reference. Twenty of the ninety sequenced cDNA clones showed strong identities with previously known sequences. The other 30 clones did not show an open reading frame in all three reading frames but the 3′ ends contained the complement of the poly A tail in the mRNA. We searched the EMBL DNA database at the nucleotide level for these sequences. Seven additional cDNA clones were identifiable. Based on these identities it was established that these sequence identities were in the 3′ regions of the sequences in the database and contained the last portion of the coding sequences. These identities are shown in Table I. The scores are the measurements of the degree of identity among the matched regions. It also takes into account the gaps that are created in order to match the corresponding sequences. The resulting scores for all random matches in the polynucleotides are below 80 and the true matches are always greater than 80. All identifiable matches were further examined for their accuracy using the ESEE program, Cabot and Beckenbach, “Simultaneous editing of multiple nucleic acids and protein sequences with ESEE,” Comput. Applic. Biosci. 5:233-234, 1989, incorporated herein by reference, which allows for the manual handling of the corresponding sequences, thus allowing for examination of the amino acid matches together with the nucleotide matches. SEQ. ID. NOS.: 1-93 represent those sequences which did not demonstrate identity with known amino acid sequences. SEQ. ID. NOS.: 94-119 represents those sequences which did demonstrable identity with known amino acid sequences. (See Table I.)

### Example X

### Construction of Subtracted cDNA Library for the Differentially Expressed mRNAs and Screening with Subtractive Probes of the Dark-Reared Cat Visual Cortex

A subtracted cDNA library for the 30-day old kitten visual cortex was constructed with some modification to the original procedure used for the synthesis of the subtracted probe. In order to establish a renewable source of material, we first constructed unidirectional lambda Zap cDNA libraries for both the 30-day kitten visual cortex and the adult cat visual cortex. Double stranded cDNA (dsDNA) was isolated from the 30-day kitten visual cortex cDNA library and the cDNA inserts were released by restriction digestion of the cloning sites. Single stranded DNA was purified from the adult cat visual cortex cDNA library and photobiotinylated. One microgram of digested dsDNA from the 30-day kitten visual cortex cDNA library was then hybridized twice with 10 micrograms of photobiotinylated single stranded DNA from the adult cat visual cortex cDNA library. After conjugation with streptavidin, the biotinylated fraction of the complex was removed and the remaining unhybridized double stranded cDNA inserts were cloned into the appropriate sites of the Lambda Zap vector. This procedure resulted in a 30-day old kitten visual cortex specific subtracted library.

Here the mRNA pool (20 ug) derived from the control tissue is photobiotinylated and the mRNA pool (1 ug) from the dark-reared 120 kitten visual cortex was converted to [³²P] labeled antisense cDNA using the reverse transcriptase. The antisense cDNA was hybridized to the biotinylated normally reared 120-day kitten visual cortex mRNA. During hybridization, the sequences common to both mRNA pools form hybrids while the sequences expressed in abundance and uniquely in the dark reared visual cortex do not hybridize to any biotinylated mRNA. The biotinylated sequences and their hybridized complements were then conjugated to streptavidin and removed by phenol/chloroform extraction. The remaining antisense [³²P] labeled cDNA which constitutes the subtractive probe was then used to screen the 30-day-old kitten visual cortex-specific subtracted cDNA library.

Eleven cDNA clones that hybridized uniquely to the dark reared-specific cDNA probe were partially sequenced. These sequences are represented by SEQ. ID. NOS.: 121-132.

### Example XI

### Administration of the Polynucleotides

Full length sequences are cloned into an HIV-1 construct as described in U.S. patent application Ser. No. 08/213,799. The vector is then inserted into the visual cortex of an adult feline over four months of age suffering from amblyopia. After the infusion of a therapeutically effective amount, the previously deprived eye is opened and the previously opened eye is sewn shut. The feline will subsequently recover function in the previously deprived eye.

From the foregoing, it will be evident that although specific embodiments of the invention have been described herein for the purposes of illustration, various modifications may be made without deviating from the spirit and scope of the invention.

1. A cDNA library, comprising polynucleotides isolated from a visual cortex of a kitten about 24-35 days old.

2. A cDNA library, comprising polynucleotides differentially expressed between polynucleotides isolated from a visual cortex of a kitten about 24-35 days and polynucleotides isolated from a visual cortex of an adult feline.

3. A cDNA library, comprising polynucleotides differentially expressed between polynucleotides isolated from a visual cortex of a dark-reared adult feline and polynucleotides of a visual cortex of an adult feline.

4. A cDNA library, comprising polynucleotides commonly expressed between cDNA libraries according to claim 2 and claim 3.

5. The cDNA library of claim 1, 2, or 4 wherein said kitten is about 25-30 days old.

6. The cDNA library of claim 1, 2, or 4 wherein said kitten is about 28 days old.

7. A cDNA library, comprising polynucleotides isolated from the visual cortex of a dark-reared adult feline.

8. A composition comprising an isolated polynucleotide having a sequence designated as one of: Seq. id. nos.: 1-132
or allelic variation thereof or complementary sequence thereto, or portion thereof at least 15 nucleotides in length.

9. The composition of claim 8 wherein said portion of said polynucleotide is at least 50 nucleotides in length.

10. The composition of claim 9 wherein said the portion of said polynucleotide is in the range of about 50-100 nucleotides in length.

11. A composition comprising an isolated cDNA displaying at least 90% homology with the coding region of a polynucleotide corresponding to a sequence designated as one of:
SEQ. ID. NOS.: 1-132.

12. The composition as in claim 11 wherein said homology is at least 95%.

13. The composition of claim 12 wherein said homology is at least 97%.

14. The composition of claim 11 wherein said cDNA encodes a cell membrane associated protein.

15. The composition of claim 11 wherein said cDNA encodes a neurotransmitter release and processing associated protein.

16. The composition of claim 11 wherein said cDNA encodes a cell or tissue remodeling associated protein.

17. The composition of claim 11 wherein said cDNA encodes a cytoskeletal protein.

18. The composition of claim 11 wherein said cDNA encodes polypeptides associated with mRNA transcription and processing.

19. The composition of claim 11 wherein said cDNA encodes polypeptides associated with energy, metabolism, and mitochondrial function.

20. The composition of claim 11 wherein said cDNA encodes polypeptides associated with neural plasticity.

21. The composition of claim 11 wherein said cDNA is at least 50 nucleotides in length.

22. The composition of claim 21 wherein said cDNA is about 50-100 nucleotides in length.

23. A composition comprising a human gene, said gene being capable of hybridizing to a sequence designated as any one of SEQ. ID. NOS.: 1-93, 120-132, or to a sequence complementary thereto, under hybridization conditions sufficiently stringent to require at least about 80% base pairing.

24. The human gene of claim 23 wherein said hybridization conditions are sufficiently stringent to require at least 90% base pairing.

25. The human gene of claim 24 wherein said hybridization conditions are sufficiently stringent to require at least 92% base pairing.

26. An antisense polynucleotide capable of blocking expression of a gene product of any one of the sequences of claim 23.

27. A triple helix probe capable of blocking expression of a gene product of any one of the sequences of claim 23.

28. A composition, comprising a nucleic acid molecule according to any one of claims 1 to 27, in substantially purified form.

29. A construct, comprising a vector capable of directing the expression of a nucleic acid molecule according to any one of claims 1 to 25.

30. The construct of claim 29 wherein the vector is selected from the group consisting of retrovirus, adenovirus, herpes simplex virus, and vaccinia virus.

31. The construct of claim 29 wherein the vector is selected from plasmids and amplicon vectors.

32. A composition, comprising ex vivo mammalian cells carrying a construct according to claim 29.

33. A method of treating warm-blooded animals for neurological disorders, comprising:
administering to a warm-blooded animal a therapeutically effective amount of a composition comprising a polynucleotide, according to any one of claims 1-27, in combination with a pharmaceutically acceptable carrier or diluent such that said neurological disorder is treated.

34. The method of claim 33 wherein said neurological disorder is selected from the group consisting of Alzheimer's disease, depression, manic depression, ischemic brain disease, epilepsy, schizophrenia, Parkinson's disease, multiple sclerosis, amyotrophic lateral sclerosis, and AIDS neurodegeneration.

35. The method of claim 33 wherein said neurological disorder is selected from the group consisting of stroke, traumatic head injury, and traumatic spinal cord injury.

36. A method of treating warm-blooded animals for learning disorders, comprising administering to a warm-blooded animal a therapeutically effective amount of a composition comprising a polynucleotide according to any one of claims 1-27 in combination with a pharmaceutically acceptable diluent or carrier, such that the learning disorder is treated.

37. A method of enhancing learning and memory of warm-blooded animals, comprising administering an effective amount of a polynucleotide according to any one of claims 1-27 in combination with a pharmaceutically acceptable carrier or diluent, such that learning and memory are enhanced.

38. The method of claims 33, 34, 35, 36 or 37 wherein the composition is administered via ex vivo mammalian cells carrying a construct according to claim 29.

39. A pharmaceutical composition, comprising any one of the polynucleotides according to claims 1-27 in a pharmaceutically acceptable diluent or carrier.

40. A composition, comprising a peptide of at least about 10 amino acids in length encoded by a sequence designated as one of:
SEQ. ID. NOS.: 1-93 and 127-132.

41. A composition as in claim 40 wherein said peptide is encoded by a sequence designated as one of SEQ. ID. NOS.: 1-10.

42. A composition as in claim 40 wherein said peptide is encoded by a sequence designated as one of SEQ. ID. NOS.: 11-20.

43. A composition as in claim 40 wherein said peptide is encoded by a sequence designated as one of SEQ. ID. NOS.: 21-30.

44. A composition as in claim 40 wherein said peptide is encoded by a sequence designated as one of SEQ. ID. NOS.: 31-40.

45. A composition as in claim 40 wherein said peptide is encoded by a sequence designated as one of SEQ. ID. NOS.: 41-50.

46. A composition as in claim 40 wherein said peptide is encoded by a sequence designated as one of SEQ. ID. NOS.: 51-60.

47. A composition as in claim 40 wherein said peptide is encoded by a sequence designated as one of SEQ. ID. NOS.: 61-70.

48. A composition as in claim 40 wherein said peptide is encoded by a sequence designated as one of SEQ. ID. NOS.: 71-80.

49. A composition as in claim 40 wherein said peptide is encoded by a sequence designated as one of SEQ. ID. NOS.: 81-90.

50. A composition as in claim 40 wherein said peptide is encoded by a sequence designated as one of SEQ. ID. NOS.: 91-93.

51. A composition as in claim 40 wherein said peptide is encoded by a sequence designated as one of SEQ. ID. NOS.: 121-126.

52. A composition as in claim 40 wherein said peptide is encoded by a sequence designated as one of SEQ. ID. NOS.: 127-132.

53. The composition of claim 40 wherein said peptide is about 100-150 amino acids in length.

54. The composition of claim 40 wherein said peptide is at least 130 amino acids in length.

55. A composition, comprising a recombinant binding partner capable of specifically binding a gene product encoded by the coded sequence designated as one of:
SEQ. ID. NOS.: 1-132
or a sequence complementary thereto.

56. The composition of claim 55 wherein said recombinant binding partner is selected from the group consisting of: antibodies or fragments thereof, peptides and small organic molecules.
